(19) United States
(12) Patent Application Publication
(10) Pub. No.: US 2022/0244259 A1
(43) Pub. Date: Aug. 4, 2022

(54) SYSTEMS AND METHODS FOR ACQUISITION AND TESTING OF BIOLOGICAL SAMPLES

(71) Applicant: Weavr Health Corp., Cambridge, MA (US)

(72) Inventors: Brandon T. JOHNSON, Cambridge, MA (US); Braden BENGE, Cambridge, MA (US); Lachlan TOBIASON, Cambridge, MA (US); Wendy HARMAN, Cambridge, MA (US); Mel HERNANDEZ, Cambridge, MA (US); Linh HOANG, Lexington, MA (US); Kate E. CHRISTIAN, Baltimore, MD (US); Aaron OPPENHEIMER, Hollis, NH (US); Taylor TOBIN, Hollis, NH (US); Jon TAYLOR, Hollis, NH (US); Brendan COLLINS, Hollis, NH (US); Art ROUSMANIERE, Hollis, NH (US)

(21) Appl. No.: 17/722,195

(22) Filed: Apr. 15, 2022

Related U.S. Application Data
(63) Continuation of application No. PCT/US2021/044236, filed on Aug. 2, 2021.
(60) Provisional application No. 63/060,279, filed on Aug. 3, 2020.
(60) Provisional application No. 63/060,292, filed on Aug. 3, 2020.
(60) Provisional application No. 63/060,456, filed on Aug. 3, 2020.
(60) Provisional application No. 63/172,032, filed on Apr. 7, 2021.
(60) Provisional application No. 63/184,062, filed on May 4, 2021.

(51) Int. Cl. G01N 33/569; G01N 33/543; B01L 3/00

(52) U.S. Cl. CPC G01N 33/56983; G01N 33/54388; B01L 3/5023; G01N 2333/165; B01L 2400/0638; B01L 2400/0487; B01L 2400/0683; B01L 2200/16; B01L 2300/069

(57) ABSTRACT

Provided herein are systems and methods of collecting a biological sample. Samples may be collected using a sample collection apparatus. The sample collection apparatus may be used to store samples for shipping. The sample collection apparatus may be used to store samples for later analysis. The sample collection apparatus may comprise a membrane having one or more test regions for detecting one or more target analytes within the sample. The sample collection apparatus may comprise a membrane having one or more test regions for quantifying one or more target analytes within the sample.

### CROSS-REFERENCE

This application is a Continuation of International Application No. PCT/US2021/044236, filed Aug. 2, 2021, which claims the benefit of U.S. Provisional Application No. 63/060,279, filed Aug. 3, 2020, U.S. Provisional Application No. 63/060,292, filed Aug. 3, 2020, U.S. Provisional Application No. 63/060,456, filed Aug. 3, 2020, U.S. Provisional Application No. 63/172,032, filed Apr. 7, 2021, and U.S. Provisional Application No. 63/184,062, filed May 4, 2021, each of which is incorporated herein by reference in its entirety.

### BACKGROUND

Biological sample acquisition typically requires collection of a sample within a container or storage medium. To perform an analysis, the collected biological sample is typically sent to a laboratory. Results of an analysis performed on a sample could take a couple of weeks to process and inform the subject or patient of the results.

### SUMMARY

Applicants have recognized systems and methods of collecting a biological sample may facilitate analysis of target analytes in individuals and among groups of individuals. A simple to use, portable sample collection apparatus may facilitate acquirement of a biological sample for an analysis of target analytes present in a biological sample.

Provided herein are embodiments of an apparatus for detecting an analyte molecule in a biological sample, the apparatus comprising: a housing having a proximal portion and a distal portion, wherein one or both of the proximal portion and distal portion are moveable so as to transition the housing from a first configuration to a second configuration; a sample interface disposed within the housing, wherein the sample interface is configured to receive the biological sample when the housing is in the first configuration; a first conduit configured to receive the biological sample via the sample interface; a mixer fluid compartment disposed within the housing, wherein the mixer fluid compartment is configured to retain a mixer fluid, wherein the mixer fluid compartment comprises a breakable seal, and wherein the breakable seal is configured to break when the housing is transitioned from the first configuration to the second configuration; a second conduit disposed within the housing, wherein the second conduit is configured to be in fluid communication with the mixer fluid compartment when the housing is in the second configuration; and a membrane disposed within the housing, wherein the membrane is in fluid communication with the first conduit and the second conduit, so as to receive the biological sample and the mixer fluid when the housing is in the second configuration, wherein the membrane comprises a testing region configured to permit detection of the analyte molecule.

In some embodiments, the analyte molecule comprises an antibody corresponding to a pathogen. In some embodiments, the testing region comprises a first detector molecule configured to emit a first signal via the presence of the analyte molecule. In some embodiments, the testing region comprises a second detector molecule spaced apart from the first detector molecule, wherein the second detector molecule is configured to emit a second signal via the presence of a second analyte molecule. In some embodiments, the testing region comprises a third detector molecule spaced apart from the first detector molecule and the second detector molecule, wherein the third detector molecule is configured to emit a third signal via the presence of a third analyte molecule. In some embodiments, the third analyte molecule is a control. In some embodiments, each detector molecule corresponds to an antibody for a pathogen or virus. In some embodiments, the pathogen or virus comprises COVID-19. In some embodiments, each detector molecule corresponds to a variant of COVID-19. In some embodiments, the first signal is emitted within 10 minutes of transitioning the apparatus from the first configuration to the second configuration when the biological sample received by the sample interface contains the analyte molecule.

In some embodiments, the second conduit comprises a hollow tube configured to rupture the breakable seal when the housing is moved from the first configuration to the second configuration. In some embodiments, the membrane comprises a capture molecule configured to bind to said analyte molecule prior to the sample flowing to the testing region, to permit detection of the analyte molecule in the testing region. In some embodiments, the apparatus further comprises a filter disposed within the first conduit. In some embodiments, said filter prevents the biological sample from reaching a distal portion of the first conduit in the first configuration.

In some embodiments, the first conduit comprises a capillary tube. In some embodiments, the apparatus further comprises a filter disposed within the first conduit. In some embodiments, said filter prevents the biological sample from reaching a distal portion of the first conduit. In some embodiments, the filter permits metering of the biological sample received within the first conduit when the housing is in the first configuration. In some embodiments, the biological sample received within the first conduit is from about 5 microliters (μL) to about 500 μL.

In some embodiments, the apparatus further comprises a sample window, wherein the sample window allows visual inspection of the biological sample within the first conduit. In some embodiments, the apparatus further comprises a testing window, wherein the testing window allows visual inspection of the testing region.

In some embodiments, the apparatus further comprises the locking tab engages a portion of the housing in the second configuration to prevent movement from the second configuration towards the first configuration. In some embodiments, the apparatus further comprises a removeable clip for preventing transition of the apparatus to the second configuration. In some embodiments, the removable clip comprises a protrusion corresponding to a recess provided in the housing, wherein mating of the protrusion within the recess facilitates retention of the removable clip on the housing. In some embodiments, a mixer plunger disposed within the housing is configured to dispense the mixing fluid into the second conduit as the housing is transitioned from the first configuration to the second configuration.

In some embodiments, i) a sample plunger disposed within the housing, ii) capillary action via the first conduit, or iii) both, facilitates movement of the biological sample through the first conduit. In some embodiments, the sample plunger is configured to move the biological sample through the first conduit as the housing is transitioned from the first configuration to the second configuration. In some embodiments, a sample plunger disposed within the housing is configured to move the biological sample through the first conduit as the housing is transitioned from a first configuration to a second configuration, and wherein a mixer plunger disposed within the housing is configured to dispense the mixing fluid into the second conduit as the housing is transitioned from the first configuration to the second configuration. In some embodiments, the sample plunger and the mixer plunger are offset, such that the biological sample is dispensed onto the membrane prior to the mixing fluid being dispensed onto the membrane. In some embodiments, the mixer fluid facilitates the biological sample to flow across the membrane to the testing region.

In some embodiments, the second conduit comprises a bend to direct the mixer fluid onto the membrane. In some embodiments, the mixer fluid compartment comprises a first mixer fluid and a second mixer fluid. In some embodiments, the apparatus comprises a first mixer plunger and a second mixer plunger disposed within the housing, wherein the first mixer fluid is separated from the second mixer fluid by the first mixer plunger. In some embodiments, the first mixer fluid and the second mixer fluid are disposed onto the membrane sequentially. In some embodiments, the mixer fluid compartment comprises a blister pack. In some embodiments, the mixer fluid compartment comprises a breakable ampule. In some embodiments, the breakable seal comprises a pierceable membrane. In some embodiments, said breakable seal is punctured by said second conduit as the housing is transitioned from a first configuration to a second configuration, thereby providing fluid communication between the second conduit and the mixer fluid compartment. In some embodiments, the mixer fluid compartment comprises a fixed, predetermined amount of mixer fluid therein. In some embodiments, the mixer fluid comprises a buffer solution, a reagent, or both. In some embodiments, one or both of the proximal portion and distal portion are moveable along a longitudinal plane of the housing. In some embodiments, one or both of the proximal portion and distal portion are configured to slide along the longitudinal plane.

Provided herein are embodiments of a method for detecting an analyte molecule in a biological sample, the method comprising: providing an apparatus comprising: a housing having a proximal portion and a distal portion, wherein one or both of the proximal portion and distal portion are moveable so as to transition the housing from a first configuration to a second configuration; a sample interface located between the proximal portion and distal portion, wherein the sample interface is configured to receive the biological sample when the housing is in the first configuration; a first conduit configured to receive the biological sample via the sample interface; a testing region disposed within the housing, wherein the testing region is in fluid communication with the first conduit; a mixer fluid compartment disposed within the housing, wherein the mixer fluid compartment is configured to retain a mixer fluid, wherein the mixer fluid compartment comprises a breakable seal, and wherein the breakable seal is configured to break when the housing is transitioned from the first configuration to the second configuration; a second conduit disposed within the housing, wherein the second conduit is configured to be in fluid communication with the mixer fluid compartment when the housing is in the second configuration; and a testing region disposed within the housing, wherein the testing region is in fluid communication with the first conduit and the second conduit, receiving the biological sample via the sample interface; and moving the housing from the first configuration to the second configuration, thereby enabling both the mixer fluid and the biological sample to be transferred to the testing region, wherein the testing region is configured to permit detection of the analyte molecule.

In some embodiments, the testing region comprises a first detector molecule configured to emit a first signal via the presence of the analyte molecule. In some embodiments, the testing region comprises a second detector molecule spaced apart from the first detector molecule, wherein the second detector molecule is configured to emit a second signal via the presence of a second analyte molecule. In some embodiments, the testing region comprises a third detector molecule spaced apart from the first detector molecule and the second detector molecule, wherein the third detector molecule is configured to emit a third signal via the presence of a third analyte molecule. In some embodiments, the third analyte molecule is a control. In some embodiments, the first signal is emitted within 10 minutes of transitioning the apparatus from the first configuration to the second configuration when the biological sample received by the sample interface contains the analyte molecule. In some embodiments, said mixer fluid processes the biological sample to permit detection of the analyte molecule. In some embodiments, the method further comprises placing the apparatus on a flat surface. In some embodiments, said flat surface is approximately perpendicular to a gravitational force. In some embodiments, the apparatus is placed on the flat surface prior to moving the housing from the first configuration to the second configuration. In some embodiments, the apparatus is placed on the flat surface prior to receiving the biological sample via the sample interface.

In some embodiments, the method further comprises removing a clip from the housing prior to moving the housing from the first configuration to the second configuration. In some embodiments, the method further comprises removing a clip from the housing prior to receiving the biological sample via the sample interface.

Provided herein are embodiments of an apparatus for collecting a biological sample, the apparatus comprising: a housing having a proximal portion and a distal portion, wherein one or both of the proximal portion and distal portion are moveable so as to transition the housing from a first configuration to a second configuration; a sample interface disposed within the housing and configured to receive the biological sample when the housing is in the first configuration; a membrane comprising a testing region, a storage region, or both; a first tube configured to receive the biological sample via the sample interface and in fluid communication with the membrane; and a mixer fluid compartment disposed within the housing and located proximal to the sample interface, and configured to retain a mixer fluid, the mixer fluid compartment having a breakable seal; wherein breakable seal is ruptured when changing the housing from the first configuration to the second configuration, such that the first tube is configured to receive the mixer fluid, and thereby displace the biological sample onto the testing region and/or the storage region.

In some embodiments, the first tube is configured to rupture the breakable seal. In some embodiments, the mixer fluid compartment is at a higher pressure than the first tube prior to the breakable seal being ruptured. In some embodiments, changing the housing from the first configuration to the second configuration provides positive displacement pressure for the mixer fluid to move within the first tube. In some embodiments, an airtight seal is provided between the proximal and distal portion of the housing. In some embodiments, the airtight seal is formed by an O-ring. In some embodiments, the O-ring is provided on an outer surface of the distal portion of the housing and contacts an inner surface of the proximal portion of the housing. In some embodiments, the first tube is located on a support structure, and wherein the first tube and support structure are configured to be at least partially placed within the mixer fluid compartment when the housing changes to the second configuration.

In some embodiments, the mixer fluid compartment is located at the proximal portion of the housing. In some embodiments, the proximal portion and the distal portion are removably coupled. In some embodiments, in the first configuration, the proximal portion is separated from the distal portion. In some embodiments, the testing region is configured to receive both the biological sample and the mixer fluid in the second configuration, wherein the testing region comprises a first detector molecule configured to emit a signal via the presence of an analyte molecule in the biological sample.

In some embodiments, the apparatus further comprises a filter disposed within the first tube. In some embodiments, the first tube comprises a capillary tube. In some embodiments, the apparatus further comprises a sample window, wherein the sample window allows visual inspection of the biological sample within the first tube. In some embodiments, the apparatus further comprises a storage window, wherein the storage window allows visual inspection of the storage and/or testing region. In some embodiments, the apparatus further comprises a locking tab, wherein the locking tab engages a portion of the housing in the second configuration to prevent movement from the second configuration towards the first configuration. In some embodiments, the apparatus further comprises a removeable clip for preventing transition of the apparatus to the second configuration. In some embodiments, the removable clip comprises a protrusion to correspond to a recess provided in the housing, wherein mating of the protrusion within the recess facilitates retention of the removable clip on the housing.

In some embodiments, the mixer fluid compartment comprises a blister pack. In some embodiments, the mixer fluid compartment comprises a breakable ampule. In some embodiments, the breakable ampule comprises glass. In some embodiments, the mixer fluid compartment comprises a membrane. In some embodiments, the membrane is ruptured by the first tube.

Provided herein are embodiments of a method for reducing the risk of a pathogen spread in a designated area, the method comprising: receiving a first biological sample from a first subject; detecting for a presence of an antibody corresponding to the pathogen; identifying the first subject as i) pathogen resistant if the presence of the antibody is detected or ii) pathogen non-resistant if the antibody is not detected; repeating (a)-(c) one or more times with one or more different subjects, wherein the one or more different subjects and the first subject together represent a total number of subjects allowed entry into the designated area; limiting the number of pathogen non-resistant subjects allowed into the designated area based at least in part on a maximum number of pathogen non-resistant subjects or a maximum proportion of the pathogen non-resistant subjects relative to the total number of subjects.

In some embodiments, the detecting of the biological sample of any one subject of the total number of subjects comprises: providing an apparatus comprising: a housing having a proximal portion and a distal portion, wherein one or both of the proximal portion and distal portion are moveable so as to move the housing from a first configuration to a second configuration; a sample interface located between the proximal portion and distal portion, and configured to receive the biological sample when the housing is in the first configuration; a first conduit configured to receive the biological sample via the sample interface and in fluid communication with a testing region; a mixer fluid compartment located between the proximal portion and the distal portion, and configured to retain a mixer fluid, the mixer fluid compartment having a breakable seal; a second conduit configured to be in fluid communication with the mixer fluid compartment in the second configuration, wherein the breakable seal is configured to break when moving the housing from the first configuration to the second configuration; and a testing region in fluid communication with the first conduit and the second conduit, so as to receive both the biological sample and the mixer fluid in the second configuration, receiving the biological sample from the any one subject via the sample interface; and moving the housing from the first configuration to the second configuration, thereby enabling both the mixer fluid and the biological sample to be transferred to the testing region, wherein the testing region comprises a first detector molecule configured to emit a signal via the presence of the analyte molecule, so as to detect for an analyte molecule within the testing region.

In some embodiments, detecting the biological sample for the any one subject is performed within 10 minutes of receiving the respective biological sample. In some embodiments, the method further comprises storing the identification of each subject as pathogen resistant or pathogen non-resistant on a computing apparatus. In some embodiments, the computing apparatus tracks the number of pathogen non-resistant subjects within the designated areas.

In some embodiments, the computing apparatus outputs the tracked number of pathogen non-resistant subjects onto a display. In some embodiments, the method further comprises identifying the individuals leaving the designated area as pathogen resistant or pathogen non-resistant. In some embodiments, the computing apparatus determines the number of pathogen non-resistant subjects in the designated area in real time based on the subjects entering and/or leaving the designated area.

Provided herein are embodiments of a method for analyte detection, comprising: providing an apparatus comprising a housing comprising (1) a sample collection region comprising a biological sample having or suspected of having an analyte, (2) a testing region in fluid communication with said sample collection region, wherein said testing region receives said biological sample or a derivative thereof from said sample collection region, and (3) a mixer fluid compartment that is fluidically isolated from said testing region, wherein said mixer fluid compartment comprises a mixer fluid, wherein said housing comprises a first portion and a second portion, one or both of which are movable relative to one another; and subjecting said proximal portion and said distal portion to movement relative to one another, wherein upon movement of said proximal portion and said distal portion relative to one another, (i) said mixer fluid compartment comes in fluid communication with said testing region to dispense said mixer fluid from said mixer fluid compartment to said testing region to permit detection of said analyte in said testing region.

In some embodiments, the mixer fluid comprises a buffer solution, a reagent, or both. In some embodiments, said mixer fluid is used to process said biological sample to permit detection of said analyte in said testing region. In some embodiments, the testing region comprises a first detector molecule configured to emit a first signal via the presence of the analyte molecule. In some embodiments, the testing region comprises a second detector molecule spaced apart from the first detector molecule, wherein the second detector molecule is configured to emit a second signal via the presence of a second analyte molecule. In some embodiments, the testing region comprises a third detector molecule spaced apart from the first detector molecule and the second detector molecule, wherein the third detector molecule is configured to emit a third signal via the presence of a third analyte molecule. In some embodiments, the third analyte molecule is a control. In some embodiments, the first signal is emitted within 10 minutes of transitioning the apparatus from the first configuration to the second configuration when the biological sample received by the sample interface contains the analyte molecule.

In some embodiments, the method further comprises placing the apparatus on a flat surface. In some embodiments, said flat surface is approximately perpendicular to a gravitational force. In some embodiments, the apparatus is placed on the flat surface prior to subjecting said proximal portion and said distal portion to movement relative to one another.

In some embodiments, the apparatus is placed on the flat surface prior to receiving the biological sample. In some embodiments, the method further comprises removing a clip from the housing prior to subjecting said proximal portion and said distal portion to movement relative to one another. In some embodiments, the method further comprises removing a clip from the housing prior to receiving the biological.

Provided herein are embodiments of an apparatus for collecting a biological sample, the apparatus comprising: a first housing comprising: a sample interface for receiving the biological sample; and a first conduit in fluid communication with the sample interface and configured to receive the biological sample via the sample interface; a second housing comprising a receiver and a base, wherein the base comprises an opening for receiving the first housing, such that the first housing is configured to at least partially be inserted within the receiver; and a membrane disposed within the base of the second housing, wherein the membrane is in fluid communication with the conduit when the first housing is inserted within the receiver, to permit movement of the biological sample from within the first conduit to a collection region located on the membrane.

In some embodiments, the first conduit is a capillary tube. In some embodiments, the first conduit comprises a first breakable seal to contain the biological sample prior to being inserted within the receiver. In some embodiments, the second housing comprises a first protrusion within the base to rupture the first breakable seal. In some embodiments, the first housing further comprises a sample holding housing configured to receive and contain the biological sample from the first conduit, wherein the sample holding housing is in fluid communication with the membrane after the first housing is inserted within the receiver. In some embodiments, the apparatus further comprises a second conduit within the second housing, wherein the first conduit is in fluid communication with the membrane via the second conduit. In some embodiments, a reagent is disposed within the first conduit or the second conduit. In some embodiments, the reagent comprises heparin, EDTA, or both.

In some embodiments, the first housing further comprises a mixer fluid compartment configured to retain a mixer fluid. In some embodiments, the mixer fluid compartment is configured to be in fluid communication with the membrane after the first housing is inserted within the receiver. In some embodiments, the mixer fluid compartment comprises a second breakable seal. In some embodiments, the second housing comprises a second protrusion within the receiver configured to rupture the second breakable seal when first housing is inserted within the receiver. In some embodiments, the mixer fluid comprises a buffer solution, a second reagent, or both. In some embodiments, rupturing the second breakable seal permits the mixer fluid to move the biological sample across the membrane.

In some embodiments, the membrane comprises a testing region configured to permit detection of an analyte molecule within the biological sample. In some embodiments, the analyte molecule comprises an antibody corresponding to a pathogen. In some embodiments, the testing region comprises a first detector molecule configured to emit a first signal via the presence of the analyte molecule. In some embodiments, the testing region comprises a second detector molecule spaced apart from the first detector molecule, wherein the second detector molecule is configured to emit a second signal via the presence of a second analyte molecule. In some embodiments, the testing region comprises a third detector molecule spaced apart from the first detector molecule and the second detector molecule, wherein the third detector molecule is configured to emit a third signal via the presence of a third analyte molecule. In some embodiments, the third analyte molecule is a control. In some embodiments, each detector molecule corresponds to an antibody for a pathogen or virus. In some embodiments, the pathogen or virus comprises COVID-19 In some embodiments, detector molecule corresponds to a variant of COVID-19. In some embodiments, the first signal is emitted within 10 minutes of transitioning the apparatus from the first configuration to the second configuration when the biological sample received by the sample interface contains the analyte molecule. In some embodiments, the first housing is separable from the second housing. In some embodiments, the base comprises a longitudinal axis, wherein the first housing is inserted within the receiver along lateral axis substantially perpendicular to the longitudinal axis of the base.

Provided herein are embodiments of an apparatus for collecting a biological sample, the apparatus comprising: a housing comprising: a first portion having a proximal portion and a distal portion, a sample interface disposed on the first portion; and a first conduit disposed within the first portion and in fluid communication with the sample interface; and a membrane disposed within a second portion of the housing, wherein the membrane is in fluid communication with the first conduit, wherein the membrane is configured to store the biological sample, test the biological sample, or both; and a cap configured to receive the first portion of the housing, such that the first portion is at least partially inserted within the cap at the proximal portion, wherein an inner surface of the cap provides an air tight or substantially air tight seal about an outer surface of the first portion of the housing, such that inserting the first portion within the cap permits air located within the cap to displace the biological sample through the first conduit and onto the membrane.

In some embodiments, the second portion comprises a longitudinal axis, wherein the first portion is inserted within the cap along a lateral axis substantially perpendicular to the longitudinal axis of the second portion. In some embodiments, the first conduit is in fluid communication with the membrane via a second conduit disposed within the second portion. In some embodiments, the apparatus further comprises a reagent disposed within the first conduit, the second conduit, and/or the membrane. In some embodiments, the reagent comprises etheylenediaminetetraacetic acid (EDTA), heparin, or both.

In some embodiments, the first conduit comprises a capillary tube. In some embodiments, the membrane comprises a testing region configured to permit detection of an analyte molecule within the biological sample. In some embodiments, the analyte molecule comprises an antibody corresponding to a pathogen. In some embodiments, the testing region comprises a first detector molecule configured to emit a first signal via the presence of the analyte molecule. In some embodiments, the testing region comprises a second detector molecule spaced apart from the first detector molecule, wherein the second detector molecule is configured to emit a second signal via the presence of a second analyte molecule. In some embodiments, the testing region comprises a third detector molecule spaced apart from the first detector molecule and the second detector molecule, wherein the third detector molecule is configured to emit a third signal via the presence of a third analyte molecule. In some embodiments, the third analyte molecule is a control. In some embodiments, each detector molecule corresponds to an antibody for a pathogen or virus. In some embodiments, the pathogen or virus comprises COVID-19. In some embodiments, each detector molecule corresponds to a variant of COVID-19. In some embodiments, the first signal is emitted within 10 minutes of transitioning the apparatus from the first configuration to the second configuration when the biological sample received by the sample interface contains the analyte molecule.

In some embodiments, the biological sample comprises a nasal fluid, an oral fluid (e.g., saliva), serum, plasma, stool, cerebrospinal fluid (CSF), urine, biopsy fluid samples, blood, or a combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1C depicts a top plan view of biological sample collection apparatus, according to some embodiments;

FIGS. 2A-2B depict a top plan view biological sample collection apparatus, according to some embodiments;

FIGS. 2C-2D depict a right side cutaway view of the biological sample collection device as depicted in FIGS. 2A-2D, according to some embodiments;

FIG. 3A depicts a perspective view of a biological sample collection apparatus adapted to receive a locking clip, according to some embodiments;

FIG. 3B depicts a right side cutaway view of the biological sample collection device as depicted in FIG. 3A, according to some embodiments;

FIG. 3C depicts a perspective view of a biological sample collection apparatus adapted to receive a locking clip, according to some embodiments;

FIG. 3D depicts a right side cutaway view of the biological sample collection device as depicted in FIG. 3C, according to some embodiments;

FIGS. 4A and 4C depict perspective views of components of a biological sample collection apparatus, according to some embodiments;

FIG. 4B depicts a right side cutaway view of a biological sample collection apparatus, according to some embodiments;

FIGS. 5A and 5B depict a top plan view of a biological sample collection apparatus, according to some embodiments;

FIG. 6A depicts an exploded view of the biological sample collection apparatus depicted in FIGS. 5A and 5B, according to some embodiments;

FIGS. 6B-6F depict a top plan cutaway view of the biological sample collection apparatus depicted in FIGS. 5A and 5B, according to some embodiments;

FIGS. 6G and 611 depict components of a biological sample collection apparatus, according to some embodiments;

FIGS. 7A and 7C depicts a top perspective cutaway view of a biological sample collection apparatus, according to some embodiments;

FIG. 7B depicts a top perspective view of a biological sample collection apparatus, according to some embodiments;

FIGS. 8A-8D depict a top cutaway view of a biological sample collection apparatus, according to some embodiments;

FIG. 9 depicts a top cutaway view biological sample collection apparatus, according to some embodiments;

FIG. 10 depicts a top cutaway view of a biological sample collection apparatus, according to some embodiments;

FIG. 11A depicts a perspective view of a biological sample collection apparatus, according to some embodiments;

FIG. 11B depicts a right side cutaway view of the biological sample collection apparatus depicted in FIG. 11A, according to some embodiments;

FIG. 11C depicts an exploded view of the biological sample collection apparatus depicted in FIG. 11A, according to some embodiments;

FIG. 12 depicts a right side cutaway view of a biological sample collection apparatus, according to some embodiments;

FIG. 13A depicts a perspective view of a biological sample collection apparatus, according to some embodiments;

FIG. 13B depicts a right side cutaway view of the biological sample collection apparatus depicted in FIG. 13A, according to some embodiments;

FIG. 13C depicts an exploded view of the biological sample collection apparatus depicted in FIG. 13A, according to some embodiments;

FIG. 14A depicts a perspective view of a biological sample collection apparatus, according to some embodiments;

FIG. 14B depicts a right side cutaway view of the biological sample collection apparatus depicted in FIG. 14A, according to some embodiments;

FIG. 14C depicts an exploded view of the biological sample collection apparatus depicted in FIG. 14A, according to some embodiments;

FIG. 15A depicts a perspective view of a biological sample collection apparatus, according to some embodiments;

FIG. 15B depicts a right side cutaway view of the biological sample collection apparatus depicted in FIG. 15A, according to some embodiments;

FIG. 15C depicts an exploded view of the biological sample collection apparatus depicted in FIG. 15A, according to some embodiments;

FIG. 16A depicts a perspective view of a biological sample collection apparatus, according to some embodiments;

FIG. 16B depicts a right side cutaway view of the biological sample collection apparatus depicted in FIG. 16A, according to some embodiments;

FIG. 16C depicts an exploded view of the biological sample collection apparatus depicted in FIG. 16A, according to some embodiments;

FIG. 17A depicts a perspective view of a biological sample collection apparatus, according to some embodiments;

FIGS. 17B-17C depict a right side cutaway view of the biological sample collection apparatus depicted in FIG. 17A, according to some embodiments;

FIG. 18A depicts a perspective view of a biological sample collection apparatus, according to some embodiments;

FIG. 18B depicts a right side cutaway view of the biological sample collection apparatus depicted in FIG. 18A, according to some embodiments;

FIG. 18C depicts distal side cutaway view of the biological sample collection apparatus depicted in FIG. 18A, according to some embodiments;

FIGS. 19A-19B depict a perspective view of a biological sample collection apparatus, according to some embodiments;

FIG. 19C depicts a proximal side cutaway view of the biological sample collection apparatus depicted in FIGS. 19A-19B, according to some embodiments;

FIG. 19D depicts right side cutaway view of a biological sample collection apparatus, according to some embodiments;

FIG. 19E depicts perspective view of the biological sample collection apparatus depicted in FIG. 19B, according to some embodiments;

FIG. 19F depicts right side cutaway view of the biological sample collection apparatus depicted in FIG. 19A, according to some embodiments;

FIG. 19G depicts a right side cutaway view of the biological sample collection apparatus depicted in FIGS. 19A-19B, according to some embodiments;

FIGS. 20A-20C depict a biological sample collection apparatus, according to some embodiments;

FIGS. 21A-21C depict a biological sample collection apparatus, according to some embodiments;

FIG. 22A depicts a mixer fluid compartment component of a biological sample collection apparatus, according to some embodiments;

FIGS. 22B-22C depict a top cutaway view of a biological sample collection apparatus, according to some embodiments;

FIGS. 23A-23D depict a biological sample collection apparatus, according to some embodiments;

FIGS. 24A-24E depict a biological sample collection apparatus, according to some embodiments;

FIG. 24F depicts a right side cutaway view of the biological sample collection apparatus of FIGS. 24A-24D, according to some embodiments;

FIG. 24G depicts an exploded view of the biological sample collection apparatus of FIGS. 24A-24D, according to some embodiments;

FIGS. 25A-25E depict a biological sample collection apparatus, according to some embodiments;

FIGS. 26A-26D depict a top plan cutaway view of a biological sample collection apparatus, according to some embodiments;

FIG. 27 depicts a perspective view of a biological sample collection apparatus, according to some embodiments;

FIGS. 28A and 28B depict a biological sample collection apparatus, according to some embodiments;

FIG. 29 depicts a biological sample collection apparatus, according to some embodiments;

FIG. 30 depicts a biological sample collection apparatus, according to some embodiments;

FIG. 31A depicts a perspective view of a biological sample collection apparatus, according to some embodiments;

FIG. 31B depicts a right side cutaway view of the biological sample collection apparatus shown in FIG. 31A, according to some embodiments;

FIG. 32 depicts a biological sample collection apparatus, according to some embodiments;

FIG. 33 depicts components of a biological sample collection apparatus, according to some embodiments;

FIG. 34 depicts components of a biological sample collection apparatus, according to some embodiments;

FIGS. 35A-35B depict components of a biological sample collection apparatus, according to some embodiments;

FIG. 36 depicts a lateral flow assay of a biological sample collection apparatus, according to some embodiments;

FIG. 37 depicts an analysis of a lateral flow assay of a biological sample collection apparatus, according to some embodiments; and

FIG. 38 depicts a computing system for performing methods disclosed herein, according to some embodiments.

### DETAILED DESCRIPTION

In some embodiments, provided herein are systems and method for acquiring a biological sample. In some embodiments, provided herein are systems and method for testing a biological sample. In some embodiments, provided herein is a sample collection apparatus or apparatus used to collect, meter, and chemically treat a biological sample. In some embodiments, the biological sample comprises a fluid collected from a patient. In some embodiments, the sample is introduced into the apparatus via a sample interface or sample interface. In some embodiments, collecting a biological fluid from a patient comprises directing blood droplets from a fingertip into a well.

In some embodiments, sample conduits receive a biological sample from the sample interface or act directly as the sample interface. In some embodiments, the sample conduits comprise capillary tubes. In some embodiments, the sample conduits act to meter a specific volume of a biological sample. In some embodiments, the sample conduits are coated with one or more reagents. The one or more reagents may be provided to preserve or stabilize the sample. The one or more reagents may be provided to facilitate processing of the sample. In one or more embodiments, the sample conduits are coated with heparin, EDTA, or both

In some embodiments, a metered sample is deposited onto a membrane via capillary action, pressure, or a plunger. In some embodiments, one or more plungers, coupled to a closeable housing, dispense fluid from the sample conduits and onto the membrane. The plungers may be attached to one or more movable housing pieces, such that when the housing is moved from a first configuration to a second configuration, the plungers are forced through the capillaries. In some embodiments, the first configuration is an open configuration and the second configuration is a closed configurations. In some embodiments, the apparatus is “activated” by moving the apparatus from the first configuration to the second configuration. In some embodiments, the apparatus is configured to receive a sample when in the open configuration. For example, in some embodiments, the sample interface is able to receive a biological sample therethrough when the apparatus is in the open configuration, but is unable to receive a biological sample when the apparatus is in the closed configuration.

In some embodiments, activation of the apparatus dispenses a mixer fluid with or after the biological sample being deposited onto a membrane. In some embodiments, the mixer fluid is pre-stored in a mixer fluid chamber which may include but is not limited to a blister pack, glass ampule, sealed chamber with pierceable membrane, syringe, or a combination thereof. In some embodiments, methods of dispensing the mixer fluid include but are not limited to crushing, piercing, squeezing, and moving the seal containing the liquid. In some embodiments, the mixer fluid comprises one or more reagents. In some embodiments, the mixer fluid comprises one or more buffer solutions. In some embodiments, about 300 microliters (μL) of mixer fluid is dispensed onto the membrane. In some embodiments, 1 to 500 microliters (μL), including increments therein, of mixer fluid is dispensed onto the membrane. In some embodiments, the apparatus dispenses a mixer fluid volume of about 1 μL to about 500 μL. In some embodiments, the apparatus dispenses a mixer fluid volume of about 1 μL to about 50 μL, about 1 μL to about 100 μL, about 1 μL to about 200 μL, about 1 μL to about 300 μL, about 1 μL to about 400 μL, about 1 μL to about 500 μL, about 50 μL to about 100 μL, about 50 μL to about 200 μL, about 50 μL to about 300 μL, about 50 μL to about 400 μL, about 50 μL to about 500 μL, about 100 μL to about 200 μL, about 100 μL to about 300 μL, about 100 μL to about 400 μL, about 100 μL to about 500 μL, about 200 μL to about 300 μL, about 200 μL to about 400 μL, about 200 μL to about 500 μL, about 300 μL to about 400 μL, about 300 μL to about 500 μL, or about 400 μL to about 500 μL. In some embodiments, the apparatus dispenses a mixer fluid volume of about 1 μL, about 50 μL, about 100 μL, about 200 μL, about 300 μL, about 400 μL, or about 500 μL, including increments therein. In some embodiments, the apparatus dispenses a mixer fluid volume of at least about 1 μL, about 50 μL, about 100 μL, about 200 μL, about 300 μL, or about 400 μL. In some embodiments, the apparatus dispenses a mixer fluid volume of at most about 50 about 100 about 200 about 300 about 400 μL, or about 500 μL.

In some embodiments, the apparatus comprises a capillary tube that protrudes upwards from the housing at an angle (for example, relative to a longitudinal axis of the housing) to allow the biological sample to be collected more easily. In some embodiments, upon closing or activation of the apparatus, the capillary tube is laid flat in the housing. The capillary tube may be aligned with plungers that dispense the sample at the end of the activation action. The capillary tube may be attached to the housing by a hinge element at the base of the capillary tube. In some embodiments, the apparatus comprises a locking feature that prevents the apparatus from being prematurely activated. For example, in some embodiments, the locking feature prevents the apparatus from moving from the first configuration (e.g., open configuration), to a closed configuration (e.g., second configuration). In some embodiments, the locking feature comprises a removable clip, a tab that must be broken, a tab that is rotated to allow movement, a push button, or a combination thereof. In some embodiments, the apparatus comprises an activated lock feature that prevents the apparatus from being opened once it has been activated. The activated lock feature includes but is not limited to a ratchet, deformable latches, deformable tabs, ramps, or a combination thereof.

In some embodiments, a stabilization agent is arranged to engage the fluid as the one or more plungers dispense fluid onto the membrane. In some embodiments, the stabilization agent may be heparin and/or EDTA. The stabilization agent may be coated or deposited onto an interior of at least one of the capillaries, the plungers, and the membrane. This configuration may also include a desiccant located adjacent the membrane. A mixing region may also be located between the capillaries and the membrane, such that the stored mixer fluid is mixed with the biological sample when the housing is moved from the open position to the closed position. In some embodiments, this mixture is then applied to the membrane. In some embodiments the membrane is a lateral flow test. In some embodiments, the apparatus comprises multiple membranes. In cases where there are multiple membranes, the sample may be collected by multiple fluid pathways and dispensed in parallel or collected as one sample and split when dispensed. In some embodiments, the multiple fluid pathways comprise one or more capillaries.

In some embodiments, the housing comprises one or more windows. In some embodiments, the housing comprises a window positioned on the housing in a location such that at least a portion of the capillaries and/or biological sample are visible through the window. In some embodiments, the windows provide a visual indication that an adequate amount of the biological sample has been collected. The housing may also include one or more windows positioned in a location such that at least a portion of the membrane or lateral flow strip are visible through the window. This may allow viewing of the assay result or confirmation of sample collection.

In some embodiments, a first housing section and second housing section engage to allow movement of the housing from the open position to the closed position, activating plungers that dispense both the mixer fluid and sample. These plungers may be staggered to allow dispensing the mixer fluid and sample at different times. In some embodiments, a plunger dispenses the mixer fluid by exerting pressure on the mixer fluid chamber. This may move the chamber to contact a piercing element which is connected to a fluid pathway or conduit leading to the membrane or mixing area (as described herein). In some embodiments, the conduit comprises one or more bends to direct the mixer fluid to an area on the membrane or in the mixing area. The piercing element may include but is not limited to a needle, a wicking material, and a hollow tube. In some embodiments, as movement from a first configuration to a second configuration continues to apply pressure on the mixer fluid chamber to causes a movable or compressible element in the chamber to move and force the liquid within the mixer compartment to be dispensed. In some embodiments, a hollow tube piercing element provides a conduit such that the mixer fluid moves through the piercing element to the membrane. In some embodiments, the conduit comprises one or more bends to direct the mixer fluid to an area on the membrane or in the mixing area. In some embodiments, the mixer compartment is pressurized so as to provide a positive displacement of the mixer fluid through a conduit when the chamber is pierced, wherein the conduit, membrane, and/or mixing region are at a lower pressure than the mixer compartment (prior to being pierced).

In some embodiments, activation of the apparatus moves a plunger to dispense the biological sample from the capillary tube onto the membrane or into the mixing area. In some embodiments, the two parts of the housing are oriented perpendicular to one another. In some embodiments, as the housing is closed, the sample interface is covered by the housing.

In some embodiments, for an apparatus described herein, the apparatus further comprises a vacuum chamber. In some embodiments, the vacuum chamber is a sealed chamber having a negative pressure. In some embodiments, the apparatus is configured to be activated (for example via a method described herein, such as moving from a first/open configuration to a second/closed configuration), and/or configured to be activated by piercing the vacuum chamber directly, such that, a vacuum is drawn through a sample conduit and/or mixer fluid conduit, to draw in a biological sample and/or mixer fluid to a membrane (as described herein). In some embodiments, activation of the apparatus permits the vacuum to draw in a plunger that subsequently dispenses a biological sample and/or mixer fluid onto a membrane.

In some embodiments, as the housing is closed, the interior of the housing exerts pressure on a blister pack containing a mixer fluid. In some embodiments, housing comprises a ramp to increase the pressure exerted on the blister pack. In some embodiments, when fully closed, the blister pack meets a piercing element which faces a pierceable side or surface of the blister pack, causing the liquid to exit and travel to the membrane. In some embodiments, activation of the apparatus also dispenses the sample by forcing a plunger through the capillary tube. In some embodiments, the sample is dispensed onto the membrane.

In some embodiments, an apparatus described herein comprises a dial having a sample interface configured to receive a biological sample, wherein the dial is configured to rotate so as to cover the sample interface. In some embodiments, rotating the dial permits the sample interface to be in fluid communication with a sample conduit (as described herein) and a membrane (as described herein). In some embodiments, rotating the dial enables a mixer fluid chamber within the apparatus to rupture. Accordingly, in some embodiments, rotating the dial permits the sample to flow to a membrane (as described herein), and also permits a mixer fluid within a mixer fluid chamber to be in fluid communication with a membrane.

The mixer fluid and sample may be dispensed at different locations on the membrane and at different times.

In some embodiments, the apparatus is provided in two separate parts. In some embodiments, a first housing of the apparatus has a protruding capillary tube used to collect a biological sample. In some embodiments, once collection is complete, the first housing is placed onto a second housing containing the membrane, creating a sealed chamber between the two pieces. In some embodiments, the first housing is referred to as a sampling or intake portion, and the second housing is referred to as the base or collection portion. In some embodiments, the capillary tube is open to the sealed chamber on one side and the membrane on the other. In some embodiments, upon pressing the top portion, a blister pack contained in the top portion is compressed into a piercing element, releasing the mixer fluid. In some embodiments, upon further compression, the air in the chamber created by the top and bottom parts is forced through the capillary tube, dispensing first the blood and then the mixer fluid onto the membrane. In some embodiments the mixer fluid is contained in a glass ampule that is cracked by a lever, spring, leaf spring, or other mechanism. In some embodiments, a wicking material connects the mixer fluid to the membrane or mixing area.

In some embodiments, other forms of fluid movement within an apparatus described herein (in addition to or alternate to a plunger and/or capillary flow), comprise fluid injection, fluid movement via pressure differential, fluid movement via gravity, fluid movement via desolvable thin films, fluid movement via micro-suction, or any combination thereof.

Sample

As described herein, a sample collection and/or testing apparatus described herein is configured to receive a sample for storage and/or for testing. In some embodiments, the sample comprises a nasal fluid, an oral fluid (e.g., saliva), serum, plasma, stool, cerebrospinal fluid (CSF), urine, biopsy fluid samples, blood, or a combination thereof. In some embodiments, the sample is provided directly from a subject (e.g., pricked finger to provide blood droplets, spit for saliva). In some embodiments, a tool is used to provide the sample, such as a syringe or pipette, which receives the sample from another storage location (such as a container). In some embodiments, a swab is used to collect a sample from a sample, wherein a device described herein (e.g., via a sample interface) is configured to extract the sample from the swab.

Testing

As described herein, a sample collection and/or testing apparatus described herein is configured to collect and test a biological sample. In some embodiments, the testing comprises a serology analysis for a subject. In some embodiments, an apparatus described herein enables for a point of care serology analysis to be performed for a subject. In some embodiments, the apparatus enables for a rapid at home serology analysis to be performed on a subject.

In some embodiments, an apparatus described herein enables for detecting previous infection and/or antibody response for a given pathogen and/or virus. For example, in some embodiments, an apparatus described herein is configured to perform a serology analysis (based on a received biological sample from a subject) for pathogens and viruses such as the Flu (Flu A, Flu B), HIV, COVID-19 (including the various COVID-19 variants), Lyme disease, Syphilis, or any combination thereof. In some embodiments, the serology analysis comprises detecting for antibodies (via a biological sample received) relating to a pathogen or virus. In some embodiments, an apparatus described herein is configured to detect for allergies (via a biological sample) such as food allergies, seasonal allergies, pet allergies, eczema, rhinitis, asthma, or a combination thereof. In some embodiments, an apparatus described herein is configured to detect for metabolic diseases (via a biological sample) such as diseases for lipids, A1c, cardiac troponin I, or a combination thereof.

In some embodiments, an apparatus described herein is configured to detect for biomarkers for a disease, such as those relating to liver function, kidney function, thyroid function, metabolic disease, women's health, men's health, nutritional related illnesses, respiratory diseases, autoimmune diseases, allergies, or a combination thereof.

In some embodiments, an apparatus described herein is configured to detect analytes relating to TSH, Glucose, Vitamin D, CRP, HbA1c, HDL, LDL, Triglycerides, Urea Nitrogen (BUN), Creatinine, eGFR Calculated, AST, ALT, Testosterone, Total PSA, SARS-CoV-2 (COVID 19), etc, or any combination thereof.

In some embodiments, the apparatus enables for a serology analysis and/or detection of other pathogen, disease, virus and/or allergy to be conducted from about 5 minutes to about 60 minutes (from the time of receiving a biological sample within the device). In some embodiments, the apparatus enables for a serology analysis and/or detection of other pathogen, disease, virus and/or allergy to be conducted from about 10 minutes to about 15 minutes (from the time of receiving a biological sample within the device). In some embodiments, the apparatus enables for a serology analysis and/or detection of other pathogen, disease, virus and/or allergy to be conducted from about 1 minute to about 15 minutes (from the time of receiving a biological sample within the device). In some embodiments, the apparatus enables for a serology analysis and/or detection of other pathogen, disease, virus and/or allergy to be conducted from about 30 seconds to about 45 minutes (from the time of receiving a biological sample within the device).

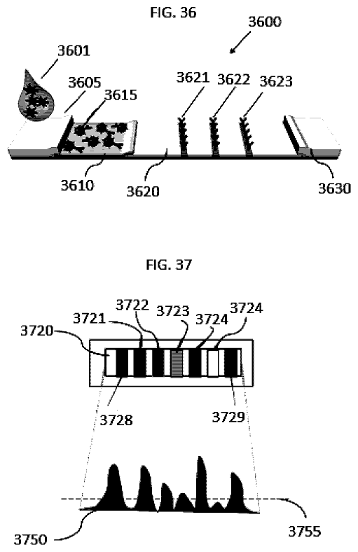

In some embodiments, as described herein for an apparatus, a membrane is provided to collect and/or test a collected biological sample. In some embodiments, as described herein, the membrane comprises a lateral flow strip to permit testing of the biological sample. In some embodiments, FIG. 36, as described herein, provides an exemplary depiction of a lateral flow strip used with an apparatus described herein. In some embodiments, other forms of testing a biological sample for an apparatus described herein include PCR, ELISA, biosensor, enzymatic reaction, immunochemiluminescent, and other forms of testing.

In some embodiments, for any apparatus described herein, the amount of biological sample collected for testing is from about 5 microliters (μL) to about 500 μL. In some embodiments, the amount of biological sample collected for storage and/or testing is from about 10 μL to about 250 μL.

### I. Lateral Activation of a Sample Collection Apparatus

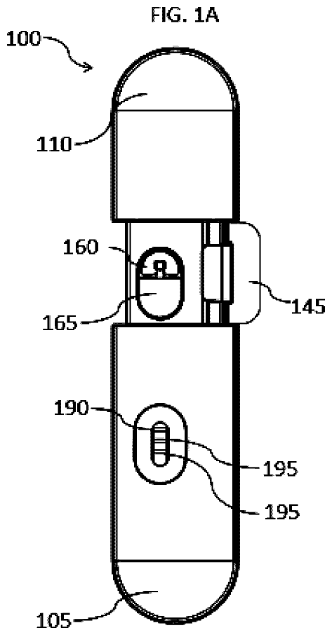

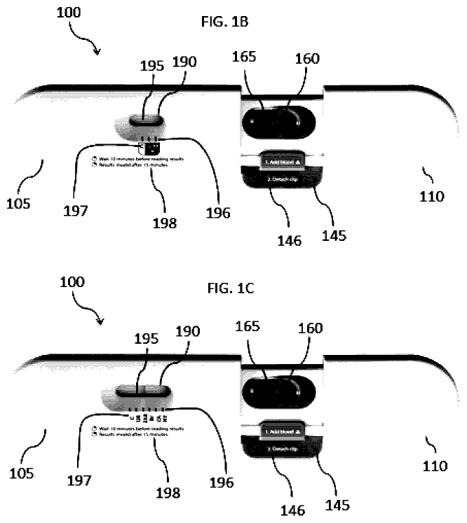

In some embodiments, with reference to FIGS. 1-10, a sample collection and/or testing apparatus is shown. In some embodiments, the apparatus is configured to be activated via axial movement of one or both of a first or proximal portion and a second or distal portion toward one another. In some embodiments, with reference to FIG. 1, a sample collection and/or testing apparatus 100 is depicted having a proximal portion 110 and a distal portion 105. In some embodiments, the apparatus 100 comprises a sample interface 160 for receiving a sample (e.g., biological sample). Sample interface 160 may also be referred to as a sample port. In some embodiments, a window 165 is provided to allow for visual inspection of a proper sample volume being deposited into the apparatus. In some embodiments, window 165 allows visualization of the sample within a capillary tube of the apparatus.

In some embodiments, the apparatus 100 (for example, see FIGS. 1A-1C) comprises a window 190 to allow for visual confirmation of a sample being deposited within a collection area of the apparatus. In some embodiments, the collection area comprises a testing region. In some embodiments, detections molecules of a specific type are applied to an area of the test region. In some embodiments, the detection molecules are provided at detection regions 195 within the test region. In some embodiments, the detection regions 195 corresponding to analytes of a different type are spaced out along the test region. In some embodiments, the housing comprises one or more markers indicating a location of correspond to a detection region 195 comprising detection molecules. In some embodiments, a first marker indicates a location of a first detection region comprising a first type of detection molecules, while a second marker indicates a location of a second detection region comprising a second type of detection molecules. In some embodiments, a marker may correspond to a control for the testing region. In some embodiments, markers are labeled to indicate the analyte being detected. For example, a marker 196 directed to a detection region for indicating the presence of a pathogen, antibody, or other target analyte. In some embodiments, multiple detection regions are provided for a plurality of variants of a target analyte or pathogen. Variants may be labeled by an abbreviation of their country of origin. In some embodiments, a marker 197 is utilized to designate a control detection region A marker may be labeled ‘P’, a marker directed to a detection region for indicating the presence of an antibody may be labeled ‘A’, or a marker directed to a detection region for indicating the presence of a control analyte may be labeled ‘C’. In some embodiments, as the sample is transferred to the testing region, results of the test (such as a lateral flow assay) are visible through the window 190. In some embodiments, the device comprises directions 198 on how to properly operate or read test results provided by the device. In some embodiments, the device comprises direction 146 printed on the clip on steps for provided a sample, detaching the clip, and/or actuating the device.

In some embodiments, each detection region comprises detection molecules that correspond to a specific analyte. Accordingly, in some embodiments, each detection region enables for the detection of a corresponding analyte that may be found in a biological sample. In some embodiments, if the specific analyte (as described herein) found within the sample is detected (for example, via the detection molecules), a signal will be emitted to indicate a presence of the analyte. In some embodiments, the signal comprises a fluorescent signal. In some embodiments, the fluorescent signal is produced by a fluorescent tag. In some embodiments, the intensity of the signal indicates the concentration of the analyte within the sample. As described herein, in some embodiments, the apparatus comprises multiple detection regions for detecting multiple analytes in a biological sample.

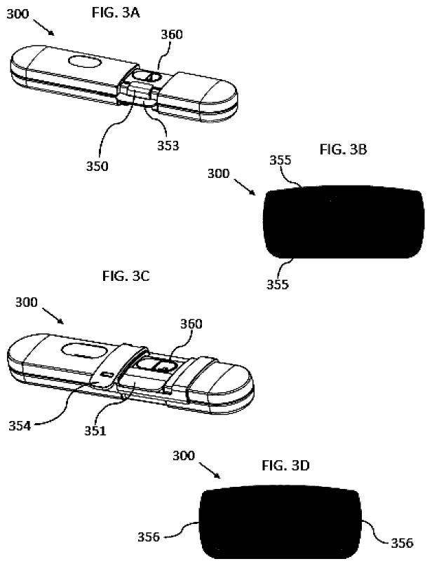

In some embodiments, the sample collection apparatus 100 comprises a clip 145. In some embodiments, the clip 145, must be removed to allow for activation of the apparatus. In some embodiments, clip 145 must be removed to allow a sample to be received by the sample interface 160. In other embodiments, as depicted in FIGS. 3A and 3C, a clip 350, 351 allows for a sample to be dispensed within the sample interface 360 while the clip is in place (prior to removal). In some embodiments, the clip (e.g., 145, 350, 351) physically prevents the apparatus from moving from an open configuration to a closed configuration (e.g., prevents activation of the apparatus).

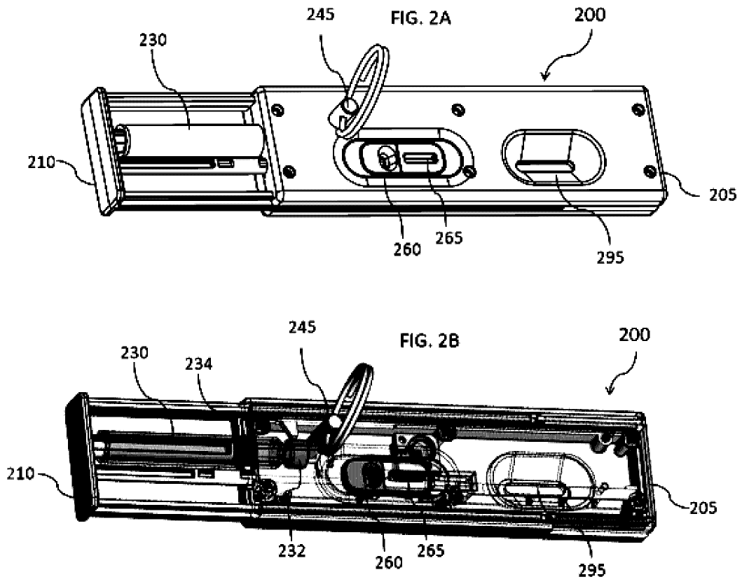

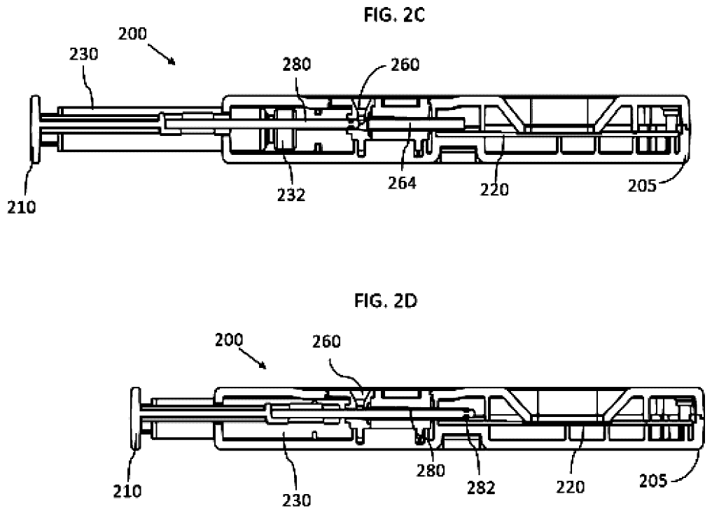

With reference to FIGS. 2A-2D, a sample collection apparatus 200 is depicted, according to some embodiments. FIG. CB depicts an apparatus in an open configuration, while FIG. 2D depicts the apparatus moving towards the closed configuration. In some embodiments, the sample collection apparatus comprises a proximal portion 210 and a distal portion 205. In some embodiments, the sample collection apparatus 200 comprises a sample interface 260 for receiving a sample. In some embodiments, a window 265 is provided to allow for visual inspection of a sufficient sample volume being deposited into the apparatus. In some embodiments, window 265 allows visualization of sample within a sample conduit 264 of the apparatus.

In some embodiments, the sample collection apparatus 200 comprises membrane 220 for collecting a sample. In some embodiments, the membrane comprises a testing region to detect or quantify an analyte within the sample. In some embodiments, the apparatus 200 comprises a sample plunger 280. In some embodiments, the sample plunger 280 pushes the sample from the sample conduit 264 onto the membrane 220 during activation (e.g., moving from an open configuration to a closed configuration) of the apparatus. In some embodiments, the sample conduit 280 is a capillary tube. In some embodiments, the capillary forces pull the sample from the sample interface 260 and within the sample conduit. In some embodiments, surface tension at a distal end of the conduit keep a liquid sample from being disposed onto the membrane 220 prior to activation of the apparatus. In some embodiments, the sample conduit acts as a metering apparatus to control the volume of the sample dispensed onto the membrane.

In some embodiments, the sample plunger 280 is configured to pass through the length of the sample conduit 264 as the apparatus is activated. In some embodiments, a recess 282 at a distal end of the plunger 280 is engaged by a tab provided at the distal end of the sample conduit 264, such that after activation, the sample plunger 280 is locked into place (for example, the apparatus cannot move back to the open configuration).

In some embodiments, the sample collection apparatus 200 comprises a mixer fluid chamber or compartment 230. In some embodiments, a mixer fluid plunger 234 is provided within the mixer compartment 230, such that movement of the proximal portion 210 toward the distal end 205 moves the plunger within the chamber to dispense the mixer fluid. In some embodiments, the mixer fluid chamber comprises a cap 232. In some embodiments, the cap comprises a pierceable membrane, to allow puncturing of the cap and disposal of the mixer fluid onto the membrane. In some embodiments, a conduit provides a fluid pathway from the chamber to the membrane. In some embodiments, activation of the apparatus (i.e. movement of the proximal portion 210 toward the distal end 205) moves the mixer compartment towards into the piercing element such that the cap 232 is pierced by the piercing element.

In some embodiments, the sample collection apparatus comprises a window 295 to allow for visual confirmation of a sample being deposited within a collection area of the apparatus. In some embodiments, the collection area comprises a testing region. In some embodiments, the testing region comprises one or more detection molecules (as described herein). In some embodiments, detections molecules of a specific type are applied to an area of the test region, thereby defining a detection region. As described herein, each detection region comprises corresponding detection molecules of a same type. In some embodiments, markers 2indicate one or more detection region comprising detection molecules of the same type. In some embodiments, as the sample is transferred to the testing region, results of the test (such as a lateral flow assay) are visible through the window 295, wherein analytes within a sample corresponding to a detection molecule in a detection region are detected (as described herein).

In some embodiments, the sample collection apparatus 200 comprises locking pin 245 to prevent accidental axial movement and activation of the apparatus. In some embodiments, as depicted in FIGS. 3A and 3C, a clip 350, 351 allows for a sample to be received within the sample interface 360 while the clip is in place (prior to removal), while preventing a housing of the apparatus from moving from an open configuration to closed configuration (e.g., preventing activation of the apparatus). In some embodiments, the clips 350, 351 comprise a tab 353, 354 to facilitate removal of the clip from the apparatus 300. In some embodiments, as depicted in FIG. 3B, the housing of the apparatus comprises recesses 355 to correspond with protrusions of the clip 350. In some embodiments, as depicted in FIG. 3D, the housing of the apparatus comprises recesses 356 to correspond with protrusions of the clip 351

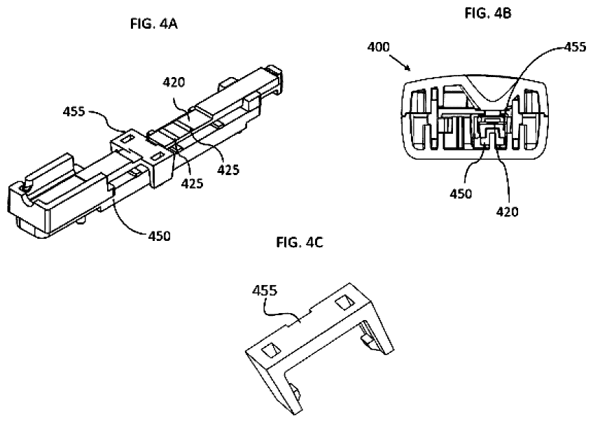

With reference to FIGS. 4A-4C, a membrane 420 within a sample collection region of a sample collection apparatus 400 described herein may be placed within a holder 450. In some embodiments, a clip 455 is used to secure the membrane 420 onto the holder 450. As disclosed herein, the membrane 420 may comprise one or more testing regions 425 for detection and/or quantification of a target analyte. The membrane may be a lateral flow assay strip.

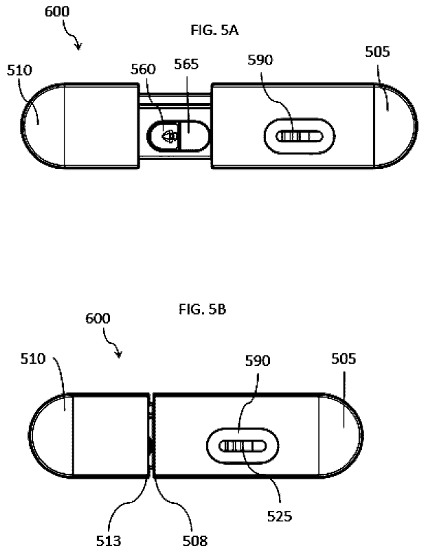

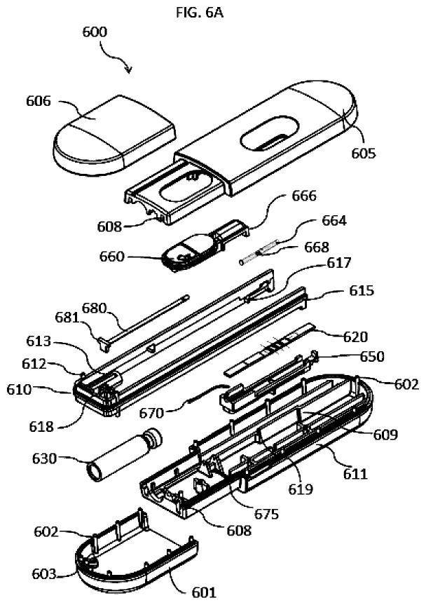

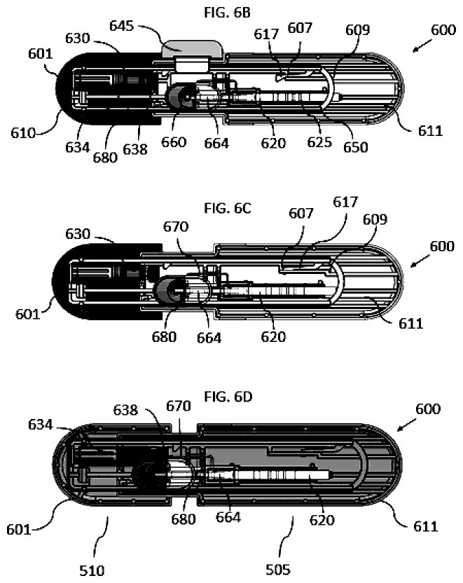

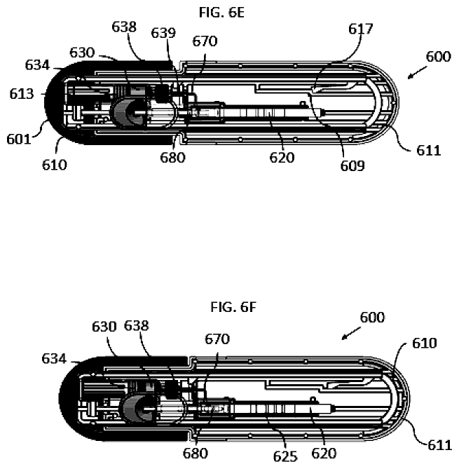

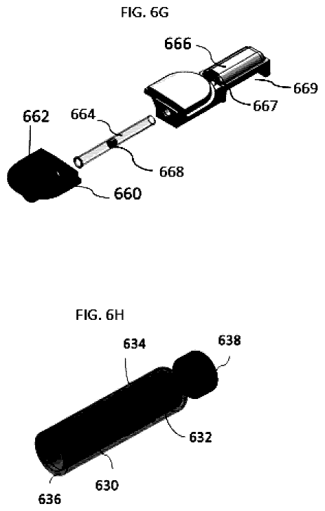

FIGS. 5A-6H depict an exemplary sample collection apparatus 600. With reference to FIGS. 5A and 5B, an axially activated sample collection apparatus is depicted, according to some embodiments. In some embodiments, FIGS. 5A-5B depicts the apparatus comprising a housing having a proximal portion 510 and a distal portion 505, one or both of which are moveable. In some embodiments, FIG. 5A depicts the sample collection apparatus in an open configuration, wherein a proximal portion of the housing 510 is spaced apart from a distal portion of the housing 505 In some embodiments, in the open configuration an upper proximal housing 606 is spaced apart from an upper distal housing 605 (see for example FIG. 6A). In some embodiments, in the open configuration, the sample interface 560 is exposed to receive a biological sample. In some embodiments, the open configuration, the sample window 565 is viewable, such that visual inspection of the sample within a sample conduit may be conducted.

In some embodiments, FIG. 5B depicts a sample collection apparatus which has been actuated (e.g., activated) such that one or both of the proximal portion 510 of the apparatus and the distal portion 505 of the apparatus have moved towards each other. In some embodiments, actuation is complete when the upper proximal housing 606 contacts or is substantially in contact with the upper distal housing 605. In some embodiments, as the apparatus is actuated, the sample is deposited onto a membrane within the apparatus. In some embodiments, as the apparatus is actuated, a buffer solution and/or a mixer fluid is deposited onto a membrane within the apparatus. In some embodiments, the membrane and the sample deposited onto the membrane are viewable through a window 590 (also referred to as a sample window or a results window). In some embodiments, wherein the membrane comprises one or more test regions 525 viewable through the window 590.

FIGS. 6A-6H depict components of the sample collection apparatus 600, according to some embodiments. In some embodiments, as described herein, the housing of the apparatus comprises a proximal portion 510 and a distal portion 505 (see FIGS. 5A-5B). In some embodiments, the proximal portion 510 comprises an upper proximal housing 606 and a lower proximal housing 601. In some embodiments, the distal portion 505 comprises an upper distal housing 605 and a lower distal housing 611. In some embodiments, the upper distal housing 605 comprises a sampling area 640 having an aperture to receive the sampling interface 660. In some embodiments, the sampling area is covered by the upper proximal housing 606 when the apparatus is configured in a closed position. In some embodiments, protrusions 602 on the lower housings 611, 601 form a friction fit with corresponding recesses (not shown) on the upper housings 605, 606. In some embodiments, the corresponding recesses and protrusions secure the lower housing components to the upper housing components. In some embodiments, an actuator 610 is disposed across at least a portion of the proximal portion 510 and the distal portion 505 (for example, see, FIGS. 6B-6F). In some embodiments, the proximal portion 510 and the actuator 610 are moveable relative to the distal portion 505. In some embodiments, the distal portion 505 is moveable relative to the proximal portion 510 and the actuator 610.

In some embodiments, actuator 610 comprises protrusions 612 to be received by recesses 603 in the upper proximal housing 606 and the lower proximal housing 601 to secure a proximal end of the actuator onto the proximal portion of the housing. In some embodiments, the actuator 610 comprises a track 615 to be received by recesses 608 in the distal portion of the housing to help guide movement of the actuator through the distal portion. In some embodiments the actuator comprises a recess 618 to receive a proximal end 681 of the sample plunger 680. In some embodiments, the actuator 610 comprises a protrusion or mixer fluid actuator 613 to abut the plunger 634 of the mixer compartment 630. In some embodiments, the actuator 610 comprises one or more locking tabs 617 to lock the apparatus in a closed or open configuration. In some embodiments, locking tab 617 comprises a flexible locking tab. In some embodiments, a force required to bend locking tab 617 and slide it over a ledge 619 provided in the distal housing must be overcome to begin actuation of the apparatus. In some embodiments, after actuation is complete, locking tab 617 locks into a recess 609 of the distal housing to lock the apparatus into a closed configuration.

In some embodiments, a sample is provided in the sample interface 660. In some embodiments, an interior surface 662 of the sample interface 660 is shaped to allow a liquid sample to flow towards the sample conduit 664 coupled with the sample interface 660. In some embodiments, the sample conduit 664 comprises a filter 668. In some embodiments, the filter removes unwanted components from the sample. In some embodiments, a mixer fluid is loaded onto the filter. In some embodiments, the conduit 664 is a capillary and the filter 668 provides a stop for capillary action of a liquid sample withing the conduit 664. In some embodiments, the filter is positioned withing the conduit to provide metering of a sample volume. In some embodiments, capillary action of the sample flows the sample past the filter and throughout the length of the conduit 664. In some embodiments, the filter is specifically positioned within the conduit to limit an amount of the sample collected within the conduit to a predetermined amount. In some embodiments, the amount of biological sample collected for storage and/or testing is from about 10 μL to about 250 μL. In some embodiments, the amount of biological sample collected for storage and/or testing is from about 5 μL to about 500 μL. In some embodiments, the conduit is received by a conduit holder 666. In some embodiments, the conduit holder 666 comprises an aperture 667 for visual inspection of the sample within the conduit 664.

In some embodiments, actuation of the apparatus comprises a user supplying a force to the ends of the housing to press the distal portion and the proximal portion together. In some embodiments, when the apparatus is actuated, the sample plunger 680 is moved through the sample conduit 664 contained within the conduit holder 666. In some embodiments, the sample plunger 680 pushes a sample through the sample conduit 664 and onto the membrane 620. In some embodiments, wherein the conduit 664 comprises a filter 668, the sample plunger 680 pushes the sample through the filter 668. In some embodiments, the plunger pushes the filter 668 out of the distal end of the conduit 664. In some embodiments, the filter, after being pushed by the plunger, remains within a region 669 between the conduit holder 666 and the surface of the membrane.

In some embodiments, an apparatus comprises as two or more sample conduits. FIG. 33 depicts an array of sample conduits 3364 comprising three sample conduits, according to some embodiments. A sample collection apparatus may comprise, for example, two, three, four, five, or six sample conduits. In some embodiments, multiple sample conduits allow for more accurate metering of a volume of a biological sample. In some embodiments, the sample conduits 3364 are coated with one or more reagents. In some embodiments, the sample conduits 3364 comprise capillary tubes. In some embodiments, each of the sample conduits comprises a filter.

In some embodiments, sample plunger 3380 comprises two or more protrusions corresponding to the two or more sample conduits 3364. A sample plunger 3880 of a sample collection apparatus may comprise, for example, two, three, four, five, or six sample protrusions to correspond to a matching number of sample conduits 3364. In some embodiments, the sample conduits 3364 receive the sample from a sample interface 3360. In some embodiments, multiple sample conduits receive the biological sample from a single sample interface. In some embodiments, conduits 3364 are held in place by a sample conduit holder 3366. In some embodiments, the sample conduit holder 3366 comprises the sample interface 3360.

In some embodiments, when the apparatus is actuated, actuator 610 pushes the mixer compartment 630 into the piercing member 670 to pierce a cap 638 of the mixer compartment. In some embodiments, piercing member 670 is a hollow member which provides a fluid conduit from the mixer compartment 630 to the membrane 620. In some embodiments, the piercing member comprises one or more bents to dispose the mixer fluid onto a proper location of the membrane 620. In some embodiments, a membrane holder 650 retains the membrane 620. In some embodiments, the mixer compartment comprises a cartridge 636 for holding a predetermined volume of mixer fluids. In some embodiments, the predetermined volume of mixer fluids is from 0 μL to about 500 μL. In some embodiments, the predetermined volume of mixer fluids is less than about 50 μL, 100 μL, 200 μL, 300 μL, or 400 μL. In some embodiments, the cartridge 636 comprises glass. In some embodiments, the cartridge 636 comprises a polymer or other suitable material which will not interfere with analysis of one or more target analytes.

With reference to FIGS. 6B-6F, a sample collection apparatus as is depicted through sequential steps of actuation or activation. In some embodiments, activation or actuation comprises a user applying a force to move a proximal portion and distal portion of the apparatus toward one another. In some embodiments, during actuation, the apparatus is changed from a first configuration (depicted in FIG. 6B) to a second configuration (depicted in FIG. 6F). In some embodiments, during actuation, the apparatus in the first configuration is an open configuration and the second configuration is a closed configuration.

FIG. 6B depicts a sample collection apparatus in an open configuration, where sample interface 660 is exposed to receive a sample. In some embodiments, the collection apparatus comprises a clip 645 to prevent accidental actuation of the apparatus. In some embodiments, a sample is provided into the sample interface 660 prior to removal of the clip 645. In some embodiments, in the open configuration, the sample plunger 680 does not obscure the entry into the sample conduit 664, such that the sample can flow into the conduit from the sample interface 660.

In some embodiments, after the sample is provided in the sample interface, the clip 645 is removed and actuation of the apparatus may begin. In some embodiments, FIG. 6C depicts a sample collection apparatus which is being actuated. In some embodiments, during the beginning stage of actuation, a distal end of the sample plunger 680 enters the sample conduit 664 and begins to push the sample out of the distal end of the sample conduit and onto the membrane 620. In some embodiments, the entry of the distal end of the sample plunger 680 into the sample conduit 664 blocks the entrance to the sample conduit and thereby prevents further sample volume from entering the apparatus. In some embodiments, locking tab 617 slides over ledge 607 and snaps back into place to prevent removal of the sample plunger 680 from the conduit 664 after the distal end of the plunger has entered the conduit. In some embodiments, bending of locking tab 617 as is slides over ledge 607 provides a slight resistance to prevent accidental actuation of the apparatus when the clip 645 is removed.

In some embodiments, FIG. 6D depicts a point during actuation at which the sample has started dispensing onto the membrane 620. In some embodiments, the mixer fluid conduit 670 comes into contact with a pierceable membrane of the mixer compartment cap 683. FIG. 6E, depicts a point of actuation at which the cap 638 contacts a stop 639 formed by a surface of the lower distal housing 611. In some embodiments, in this position, almost all the sample is dispensed out of the sample conduit and onto the membrane. In some embodiments, at the position depicted in FIG. 6E, the mixer fluid commences to dispense as the conduit 670 has pierced through the cap 38 and entered into the mixer compartment 638. In some embodiments, the protrusion 613 of the actuator 610 begins to push the mixer fluid plunger 634 toward the distal end of the mixer compartment 630, and thereby pushes the mixer fluid into the mixer fluid conduit 670 to begin dispensing the mixer fluid onto the membrane. In some embodiments, at this position, the locking tab 617 locks the apparatus in a close configuration as it snaps over ledge 609. In some embodiment, a support 675 is provided for the mixer fluid conduit 670. In some embodiments, the support is provided by an extrusion of the lower distal and/or upper distal housings.

In some embodiments, as depicted in FIG. 6F, once the sample collection apparatus reaches the closed configuration, the mixer fluids have been dispensed. In some embodiments, actuation is stopped when actuator 610 contacts a surface of the distal housing. In some embodiments, as described herein, after a period of time passes, test regions 625 of the membrane will emit a signal upon detection of a target analyte. In some embodiments, the intensity of the signal of the test regions 625 is used to quantify the concentration of analytes in the sample.

As described herein, in some embodiments, the membrane comprises a lateral flow strip configured to process a biological sample (e.g., blood molecules), wherein the biological sample is exposed to a detection region (as described herein) on the surface of the lateral flow assay strip that is configured to bind to specific analytes. In some embodiments, the analytes are bound by a conjugate which enables them to be detectable. In some embodiments, the bound analytes modify the optical or electrical properties of the surface they are bound to, making them directly detectable via visual inspection, or electronic circuits. As described herein, in some embodiments, a window in the housing provides easy access to review test and optional control lines.

In some embodiments, the lateral flow strip comprises one or more binding molecules, such as colored nanoparticles (or labels) and antibodies. In some embodiments, the binding molecules are located on a conjugate pad on the lateral flow strip. Example assays include diagnostic assays and chemical detection assays. Diagnostic assays may include, without limitation, lateral flow assays, and may include one or more serological assays. In some embodiments to perform the assay(s), a collected sample flows from the capillary tube onto a sample pad on the lateral flow strip, through a conjugate pad (on the lateral flow strip), to a membrane and from there onto an absorbent pad.

In some embodiments, the sample pad acts as a first stage. In some embodiments, the sample pad contains a filter. In some embodiments, the filter facilitates accurate and controlled flow of the sample through the rest of the apparatus. In some embodiments, the conjugate pad stores the conjugated labels and antibodies. In some embodiments, the sample exits from the sample pad and onto the conjugate pad. In some embodiments, if a target analyte is present in the sample, the immobilized conjugated antibodies and labels bind to the target and continue to flow along a membrane. In some embodiments, the membrane is a cellulose substrate. In some embodiments, binding mixer fluids situated on the membrane bind to the target at one or more test lines or detection regions on the testing region. As described herein, in some embodiments, a colored line will form at the test regions upon detection of the target analyte. In some embodiments, the density or intensity of the test line will vary depending on the quantity of the target present. In some embodiments, any remaining sample passes on to the absorbent pad to be collected. In some embodiments, the absorbent pad stores excess sample for further analysis. In some embodiments, the absorbent pad comprises one or more reagents for preserving the sample. In some embodiments, a collection apparatus further comprises a control line to confirm the apparatus is operating properly.

As described herein, in some embodiments, a membrane as described herein with any embodiment of an apparatus comprises a lateral flow strip. FIG. 36 depicts an exemplary lateral flow strip 3600 for receiving a fluid 3601 comprising one or more analytes of interest. In some embodiments, the fluid 3601 comprises a biological sample. In some embodiments, the fluid 3601 comprises a solution containing a biological sample and a mixer fluid, as disclosed herein. In some embodiments, the mixer fluid comprises one or more reagents and/or a buffer solution. In some embodiments, the lateral flow strip comprises a sample pad 3605, as disclosed herein. In some embodiments, the sample pad is configured to receive the fluid (e.g., biological sample) and/or one or more mixed fluids, from, for example, any combination of a sample interface, a mixer compartment, etc. In some embodiments, the flow strip 3600 comprises a conjugate pad 3610. In some embodiments, binding molecules 3615 are located on the conjugate pad 3610, as disclosed herein. In some embodiments, the flow strip 3600 comprises a testing region 3620 (as described herein) comprising one or more test lines or detection regions 3621, 3622, 3623, as disclosed herein. In some embodiments, the flow strip 3600 comprises the absorbent pad 3630 to collect excess fluid (e.g., biological sample) and/or mixer fluid, as disclosed herein. In some embodiments, the fluid 3601 (e.g., biological sample), is configured to flow across the lateral flow strip 3600 via the mixer fluid (e.g., buffer solution, which may comprise water, a saline solution, other types of buffer solutions). In some embodiments, the mixer fluid provides sufficient liquid volume to promote capillary flow of the biological sample (for example) across the lateral flow strip.

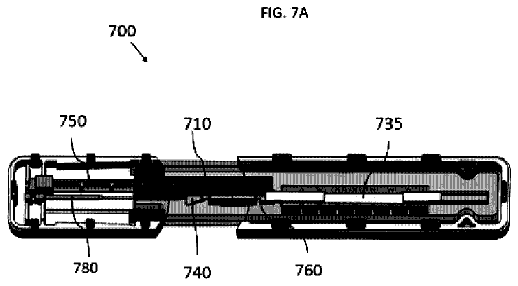

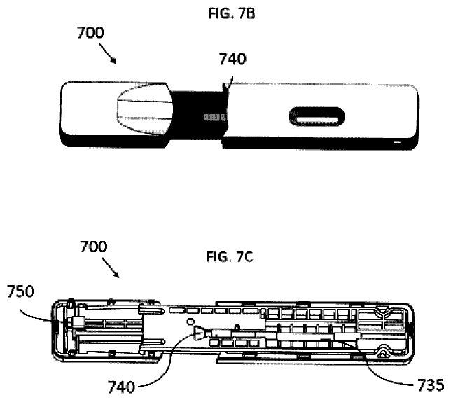

With reference to FIGS. 7A-7C, a sample collection apparatus 700 is depicted, according to some embodiments. In some embodiments, the sample collection apparatus 700 is configured to transition from an open configuration to a closed configuration via a distal portion and proximal portion, as described herein for other sample collection apparatuses. In some embodiments, the sampling apparatus comprises a mixer fluid compartment 710, a mixer plunger 750, a sample interface 740, a sample plunger 780, and a membrane 735, as disclosed herein. In some embodiments, the sample collection apparatus further comprises a mixing chamber 760 for mixing of the mixer fluid and the sample prior to disposing of the sample onto the membrane. In some embodiments, the mixing chamber comprises one or more features to facilitate mixing of the mixer fluid with the sample. In some embodiments, the membrane is as described herein, configured for collecting and in some cases, testing a sample (for example, in some cases, the membrane comprises a lateral flow strip assay).

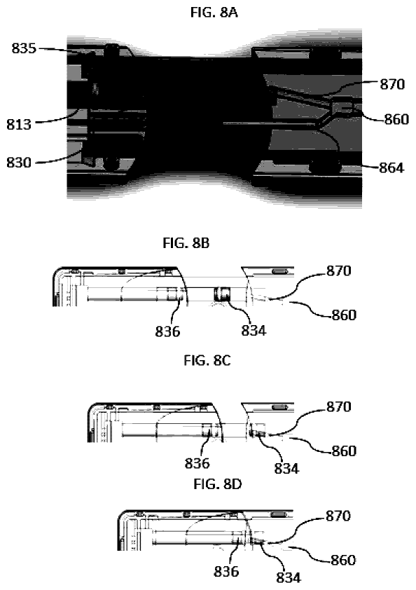

In some embodiments, as depicted in FIG. 8A, for a sample collection apparatus described herein, the sample collection apparatus comprises a sample conduit 864 and a mixer fluid conduit 870 which lead to a mixing chamber 860. In some embodiments, one or more mixer fluids are stored within a packet or ampule 835 which is crushed by a member 813 to release one or more mixer fluids into the mixer fluid compartment 830. In some embodiments, a mixer fluid packet 835 comprises a flexible bag which is compressed by the plunger to dispense a mixer fluid. In some embodiments, the member 813 comprises a seal to facilitate movement of the mixer fluids from the mixer fluid compartment 830 to the mixing chamber 860 during actuation of the apparatus. In some embodiments, a portion of the membrane is provided within the mixing chamber 860. In some embodiments, the membrane is provided within the adjacent to chamber 860. In some embodiments, the membrane is as described herein, configured for collecting and in some cases, testing a sample (for example, in some cases, the membrane comprises a lateral flow strip assay). In some embodiments, a wicking element is provided to transport the sample and a mixing fluid from the mixing chamber to the membrane.

In some embodiments, as depicted in FIGS. 8B-8D, a sample collection apparatus described herein comprises a first mixer fluid plunger 834 and a second mixer fluid plunger 836 disposed within a mixer fluid compartment. In some embodiments, a mixer fluid is provided within the mixer fluid compartment between the first and second first mixer plungers. In some embodiments. In some embodiments, an end of the mixer fluid compartment 870 is positioned at the mixer fluid compartment such that during actuation the mixer fluid is dispensed into the conduit 870 and to the mixing chamber 860. In some embodiments, the first mixer fluid plunger 834, then moves past the distal opening of the conduit 870 (as depicted in FIG. 8C). In some embodiments, as actuation continues, the mixer fluid is then dispensed into the conduit 870 and the mixing chamber 860. In some embodiments, the providing the mixer fluid between the plungers allows for the dispensing of the mixer fluid to be offset from the dispensing of a sample onto the membrane.

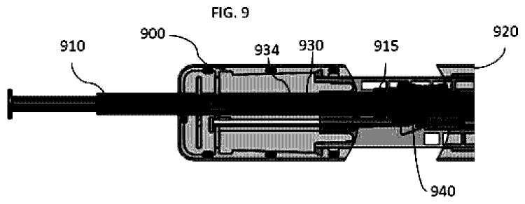

In some embodiments, as depicted in FIG. 9, a sample collection apparatus comprises a housing having a distal portion 920, a proximal portion 900, and a mixer fluid plunger 934. In some embodiments, a cap 915 comprises a luer actuated valve to retain one or more mixer fluids within a mixer fluid compartment 930. In some embodiments, an actuator 910 is configured to interface with the plunger 934. In some embodiments, the actuator 910 is coupled with the housing, and optionally detachably coupled. In some embodiments, the actuator 910 and plunger 934 are operatively coupled and move in unison. In some embodiments, the actuator 910 is configured to be inserted within the housing. In some embodiments, connector 940 interfaces with cap 915 to release the mixer fluids from chamber 930. In some embodiments, movement of distal portion 920 toward the proximal portion 900 interfaces cap 915 with connector 940. In some embodiments, such interface between the cap 915 and connector 940 allows to open a luer valve in fluid communication with a conduit that is in fluid communication with a mixing region or membrane (as described herein). In some embodiments, the housing is are moved from an open configuration to a closed configuration (as described herein, via the proximal portion and distal portion) to provide a sample to the membrane, and to allow the cap 915 and connector 940 to interface. In some embodiments, the actuator is then moved to provide a mixer fluid to the membrane. In some embodiments, the actuator is moved first to provide a mixer fluid to the membrane, then the housing is moved to a closed configuration to provide the sample to the membrane.

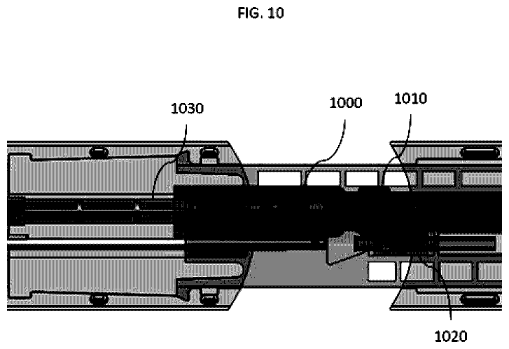

In some embodiments, as depicted in FIG. 10, for a sample collection apparatus described herein, the apparatus comprises a mixer fluid chamber 1000, a mixer fluid actuator 1030, and a mixer fluid conduit 1020. In some embodiments, the mixer fluid chamber is sealed via a cap 1010, wherein the apparatus comprising a septum defining the mixer fluid conduit. In some embodiments, the septum ruptures from a force applied to the mixer fluid actuator 1030 during actuation of the apparatus, thereby dispensing the mixer fluid within the mixer fluid chamber into the mixer fluid conduit.

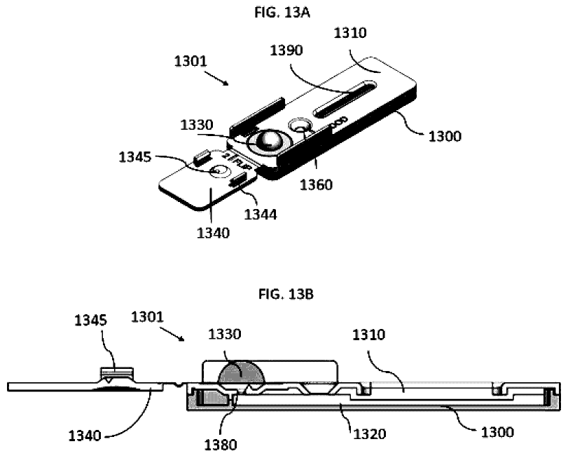

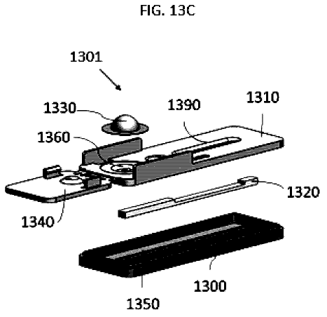

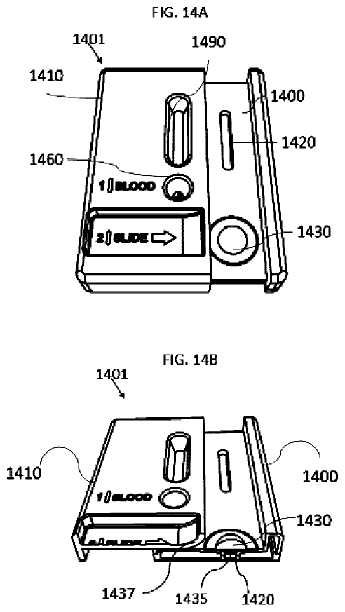

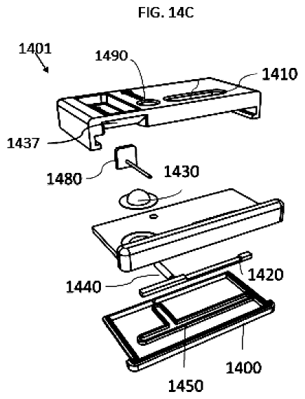

In some embodiments, as depicted in FIGS. 14A-14C, a sample collection apparatus 1401 is depicted, wherein the apparatus 1401 comprises a housing having a proximal portion 1410 and a distal portion 1400. In some embodiments, a sample is disposed in the sample interface 1460. In some embodiments, after the sample is received, the apparatus is actuated by pressing the proximal portion and distal portion together, one or both of which are moveable. In some embodiments, after the apparatus is actuated, sample plunger 1480 dispenses the sample through the sample conduit 1440 and onto the membrane 1420. In some embodiments, during actuation, a ramped portion 1437 of the proximal portion is forced over a mixer fluid chamber disposed on the distal portion 1400. In some embodiments, the mixer fluid compartment 1430 ruptures due to the contact with the ramped portion 1437, to provide one or more mixer fluids to the membrane 1420. In some embodiments, the mixer fluid helps promote the movement of the sample across the membrane, towards a collection portion and/or testing region on the membrane 1420 (as described herein). In some embodiments, the mixer fluid chamber 1430 comprises a blister pack. In some embodiments, a piercing element 1435 is provided to facilitate rupturing of the mixer fluid chamber 1430. In some embodiments, a window 1490 is provided to view the membrane 1420 after the apparatus has been actuated. In some embodiments, the membrane is as described herein, configured for collecting and in some cases, testing a sample (for example, in some cases, the membrane comprises a lateral flow strip assay).

In some embodiments, as depicted in FIGS. 15A-15C, a sample collection apparatus 1501 is depicted, wherein the apparatus 1501 comprises a proximal portion 1510 and a distal portion 1500. In some embodiments, a sample is disposed in the sample interface 1560. In some embodiments, after the sample is received, the apparatus is actuated by pressing the proximal portion and distal portion together, one or both of which are moveable. In some embodiments, the proximal portion is slides over the distal portion. In some embodiments, the proximal portion and the distal portion comprise corresponding protrusions and recesses to create a track to guide the portions when they are slid together. In some embodiments, actuating the apparatus transitions the housing from an open configuration to a closed configuration. In some embodiments the actuating the apparatus enables the sample plunger 1580 to dispense the sample (received via the sample interface) through a sample conduit 1540 and onto the membrane 1520. In some embodiments, the apparatus 1501 further comprises a mixer fluid chamber 1530 configured to rupture to provide one or more mixer fluids to the membrane 1520. In some embodiments, the mixer fluid chamber 1530 comprises a blister pack. In some embodiments, a mixer fluid conduit 1570 is configured to be in fluid communication with the sample conduit 1540 and provides a piercing element to facilitate rupturing of the mixer chamber 1530. In some embodiments, the housing further comprises a pierceable member with the distal portion 1500, wherein when the housing transitions to a closed configuration. In some embodiments, a window 1590 is provided to view the membrane 1520 after the apparatus has been actuated. In some embodiments, a recess 1550 in the retains the membrane within the housing 1500. In some embodiments, the membrane is as described herein, configured for collecting and in some cases, testing a sample (for example, in some cases, the membrane comprises a lateral flow strip assay).

In some embodiments, as depicted in FIGS. 16A-16C, a sample collection apparatus 1601 is depicted, wherein the apparatus 1601 comprises a proximal portion 1610 and a distal portion 1600. In some embodiments, a sample is disposed in the sample interface 1660. In some embodiments, after the sample is received, the apparatus is actuated by pressing the proximal portion and distal portion together, one or both of which are moveable. In some embodiments, the proximal portion is slides over the distal portion. In some embodiments, the proximal portion and the distal portion comprise corresponding protrusions and recesses to create a track to guide the portions when they are slid together. In some embodiments, actuating the apparatus transitions the housing from an open configuration to a closed configuration. In some embodiments, actuating the apparatus enables the sample plunger 1680 to dispense the sample through the sample conduit 1640 and onto the membrane 1620. In some embodiments, the apparatus 1601 further comprises a mixer fluid chamber 1630 ruptures to provide one or more mixer fluids to the membrane 1620. In some embodiments, the mixer fluid chamber 1630 comprises a blister pack. In some embodiments, a mixer fluid conduit 1675 provides the mixer fluid to the sample conduit 1640 and provides a piercing element to facilitate rupturing of the sample chamber 1630. In some embodiments, a window 1690 is provided to view the membrane 1620 after the apparatus has been actuated. In some embodiments, a recess 1650 in the retains the membrane within the housing 1600. In some embodiments, the membrane is as described herein, configured for collecting and in some cases, testing a sample (for example, in some cases, the membrane comprises a lateral flow strip assay).

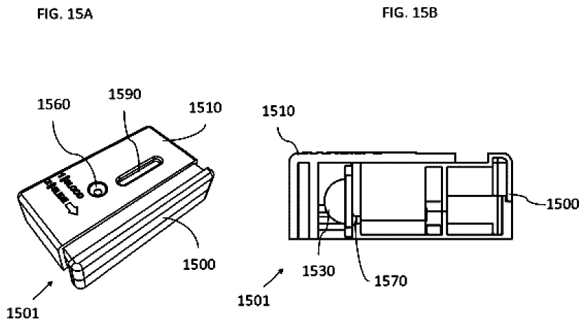

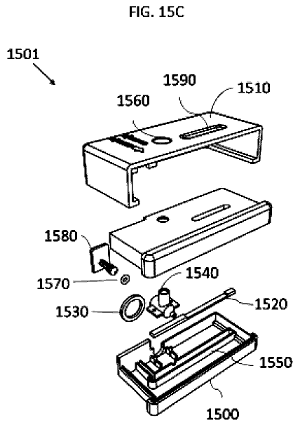

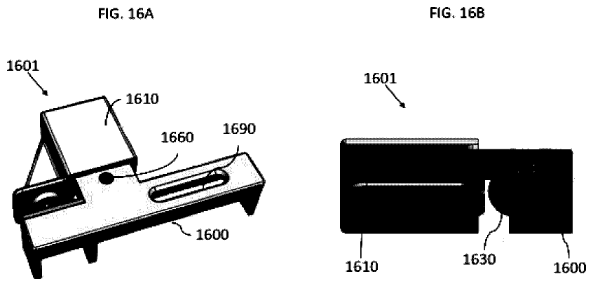

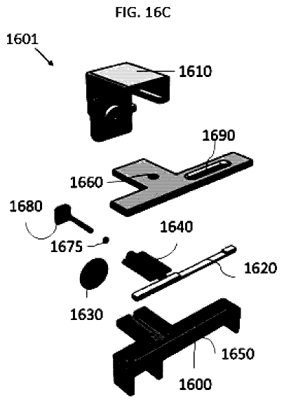

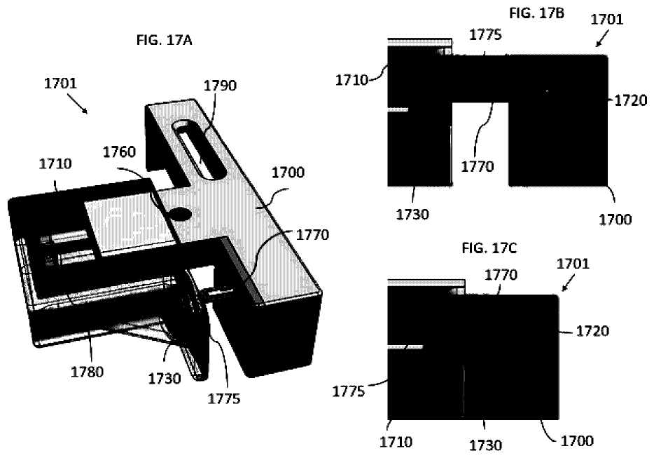

In some embodiments, as depicted in FIGS. 17A-17C, a sample collection apparatus 1701 is depicted, wherein the apparatus 1701 comprises a proximal portion 1710 and a distal portion 1700. In some embodiments, a sample is disposed in the sample interface 1760. In some embodiments, after the sample is received, the apparatus is actuated by pressing the proximal portion and distal portion together, one or both of which are moveable. In some embodiments, the proximal portion is slides over the distal portion. In some embodiments, the proximal portion and the distal portion comprise corresponding protrusions and recesses to create a track to guide the portions when they are slid together. In some embodiments, actuating the apparatus transitions the housing from an open configuration to a closed configuration. In some embodiments, actuating the apparatus enables the sample plunger 1780 to dispense the sample through the sample conduit 1740 and onto the membrane 1720. In some embodiments, the apparatus 1701 comprises a mixer chamber 1730 configured to rupture to provide one or more mixer fluids to the membrane 1720. In some embodiments, the mixer fluid chamber 1730 comprises a blister pack. In some embodiments, a piercing member 1775 facilitates rupturing of the mixer fluid chamber 1730. In some embodiments, the distal portion comprises the piercing member 1775. In some embodiments, transitioning the housing to a closed portion enables the piercing member to pierce the mixer fluid. In some embodiments, the piercing member comprises a wicking element 1770 configured to transport one or more mixer fluids from the mixer chamber to the membrane 1720. In some embodiment, the wicking element is coupled to the membrane to facilitate mixer fluid transport to the membrane. In some embodiments, a window 1790 is provided to view the membrane 1720 after the apparatus has been actuated. In some embodiments, the membrane is as described herein, configured for collecting and in some cases, testing a sample (for example, in some cases, the membrane comprises a lateral flow strip assay).

In other embodiments, the proximal portion 1710 is coupled to the distal portion 1700 via a hinge, or other tethered structure. In such embodiments, once a biological sample is received, the proximal portion, having a plunger and piercing structure, is disposed onto the distal portion, such that the plunger dispenses the biological sample onto the membrane, and the piercing structure permits a mixer fluid chamber to be ruptured, so as to dispense mixer fluid onto the membrane. In some embodiments, the sample interface and/or the mixer fluid chamber are disposed above the membrane, such that the proximal portion interfaces with the distal portion on a top surface thereof, so as to enable the plunger to dispenses the biological sample substantially vertically and substantially perpendicular to a longitudinal axis of a membrane.

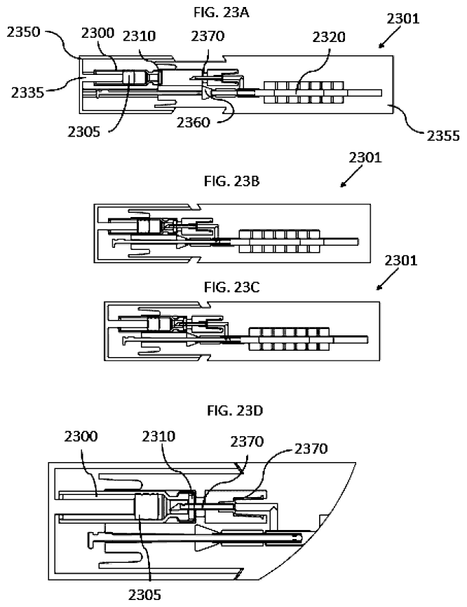

FIGS. 23A-23C depict a sample collection apparatus 2301 as it is being actuated (as described herein, wherein a housing of the apparatus 2301 transitions from an open configuration to a closed configuration, via movement of one or both of a distal portion 2355 and proximal portion 2350 of the housing). FIG. 23A depicts a sample collection apparatus in an open configuration, where sample interface 2360 is exposed to receive a sample. After the sample is provided in the sample interface, the sample collection apparatus may be actuated. In some embodiments, FIG. 23B depicts a point during actuation at which a plunger displaces the sample through a sample conduit and onto the membrane 2320. In some embodiments, the positioning of the apparatus in FIG. 23B facilitates all or almost all the sample to be dispensed onto the membrane 2320, while the mixer fluid only commences to be dispensed into a sample conduit, via a mixer fluid conduit 2370 that is configured to puncture the cap 2310 of the mixer fluid chamber 2300. In some embodiments, as depicted in FIG. 23C, once the sample collection apparatus reaches the closed configuration, an actuator 2335 engaged with a mixer fluid plunger 2305 enables all the mixer fluid to be dispensed from the mixer fluid chamber. In some embodiments, FIG. 23D depicts an additional view of the sample collection apparatus in a closed configuration, wherein plunger 2305 abuts a distal end of the mixer fluid, chamber (which is abutted by the actuator), and wherein the mixer fluid has flowed onto the membrane via the mixer fluid conduit 2370. In some embodiments, an end of the mixer fluid conduit is tapered to form a piercing member. In some embodiments, the piercing member ruptures a breakable seal provided on a cap 2310 of the mixer fluid compartment 2300. In some embodiments, the breakable seal is a septum. In some embodiments, a support 2345 is provided for the mixer fluid conduit. In some embodiment, a support 2375 is provided for the mixer fluid conduit 2370. In some embodiments, the support is provided by an extrusion of the lower distal and/or upper distal housings.

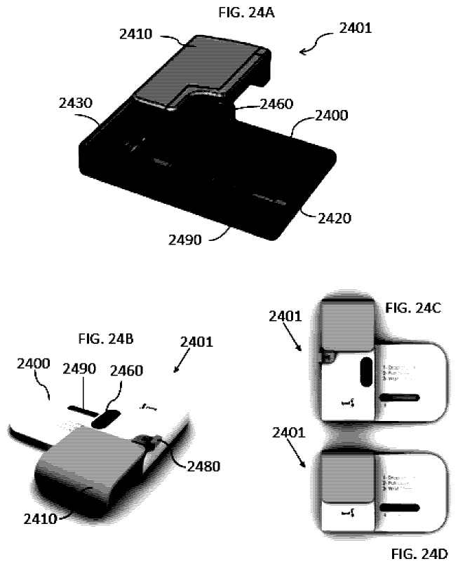

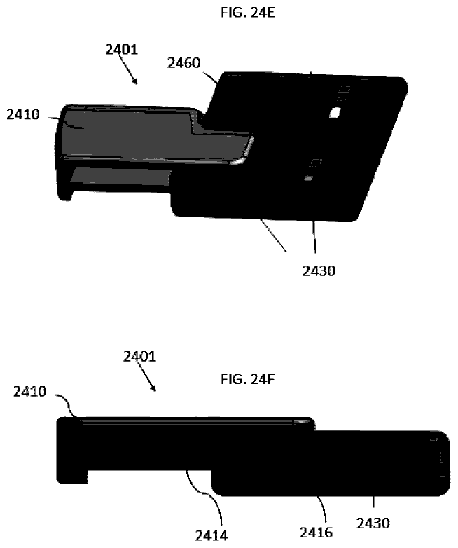

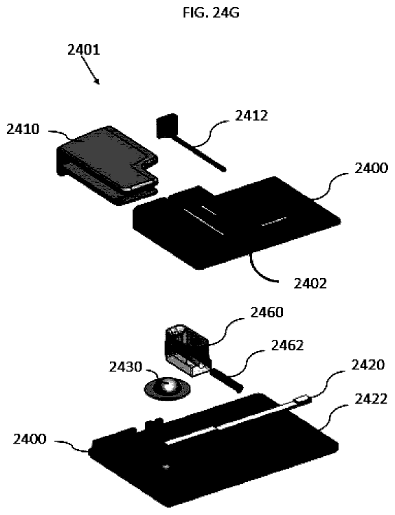

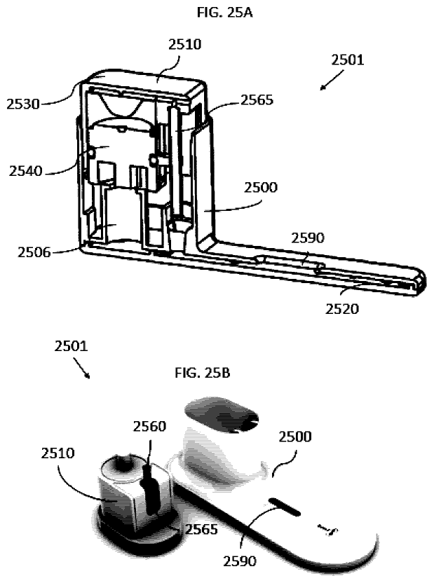

FIGS. 24A-24F depict a sample collection apparatus 2401 according to some embodiments. In some embodiments, the sample collection apparatus comprises a housing comprising a proximal portion 2410 and a distal portion 2400. In some embodiments, a sample is disposed in the sample interface 2460. In some embodiments, after the sample is received, the apparatus is actuated by moving the proximal portion and distal portion together, one or both of which are moveable. In some embodiments, the proximal portion slides over the distal portion. In some embodiments, the proximal portion and the distal portion comprise corresponding protrusions and recesses to create a track (e.g., see 2402 of FIG. 24F) to guide the portions when one or both of the proximal portion and the distal portion are moved towards each other. In some embodiments, actuating the apparatus transitions the housing from an open configuration to a closed configuration. In some embodiments, after providing a sample in the sample interface 2460, a sample conduit 2462 in fluid communication with a membrane 2420 disposed within the housing (e.g., distal portion 2400) permits the sample to be dispensed onto the membrane 2420. In some embodiments, the membrane 2420 is disposed within a membrane holder 2422 within the distal portion 2400. In some embodiments, the apparatus comprises a sample plunger 2412 configured to force the biological sample through sample conduit 2462 and onto the membrane 2420, when the apparatus 2401 transitions from an open configuration to a closed configuration. In some embodiments, the sample plunger is disposed within the proximal portion 2410. In some embodiments, the membrane is as described herein. In some embodiments, the membrane comprises a lateral flow strip, as described herein. In some embodiments, as described herein, actuating the apparatus (e.g., transitioning the housing into a closed configuration) enables a sample plunger 2412 to dispense the sample through the sample conduit 2462 onto the membrane 2420.

In some embodiments, the distal portion 2400 comprises a mixer fluid chamber holding one or more mixer fluids, as described herein. In some embodiments, the mixer fluid chamber 2430 comprises a breakable seal configured to release the one or more mixer fluids therefrom. In some, actuating the apparatus enables an actuator 2414 within the housing (e.g., proximal portion 210) that is configured to rupture the mixer fluid chamber 2430 to dispense one or more mixer fluids. With reference to FIG. 24E, in some embodiments, the actuator 2414 comprises a ramped end (e.g., 2416) that is configured to interface with the mixer fluid chamber 2430 when the housing is moved from the open configuration to the closed configuration. Accordingly, in some embodiments, the actuator 2414 is configured to rupture the mixer fluid chamber 2430 via interaction with the ramped end 2416 of the actuator 2414. In some embodiments, the actuator 2414 is configured to dispense the one or more mixer fluids, onto the membrane 2420, and in some cases, helps move the biological sample across the membrane 2420. In some embodiments, the mixer fluid chamber comprises a blister pack. In some embodiments, the membrane is provided perpendicular to the length of the sample conduit.

In some embodiments, the membrane 2420 comprises test region. In some embodiments, the membrane is as described herein, configured for collecting and in some cases, testing a sample (for example, in some cases, the membrane comprises a lateral flow strip assay). In some embodiments, a window 2490 is provided to view the test region. In some embodiments, a removable tab 2480 blocks actuation of the housing. In some embodiments, a portion of the actuator covers the sample interface in the closed configuration.

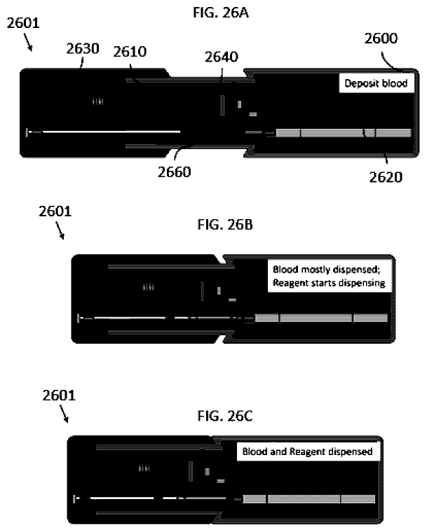

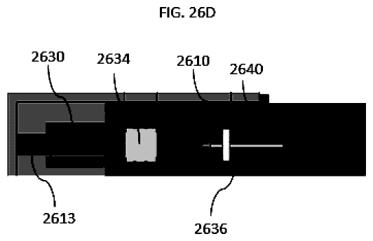

FIGS. 26A-26C depict another exemplary sample collection apparatus as it is being actuated. FIG. 26A depicts a sample collection apparatus 2601 in an open configuration, where sample interface 2660 is exposed to receive a sample. After the sample is provided in the sample interface, the sample collection apparatus may be actuated. In some embodiments, FIG. 26B depicts a point during actuation at which the sample has been dispensed onto the membrane 2620 by the sample plunger. At this time, almost all the sample should be dispensed, however the mixer fluid may just be starting to dispense as the conduit 2640 punctures the cap 2610 of mixer fluid compartment 2630. In some embodiments, as depicted in FIG. 26C, once the sample collection apparatus reaches the closed configuration, all the mixer fluid has been dispensed. In some embodiments, as depicted in FIG. 26A-26C, the mixer fluid conduit 2640 comprises a flexible tubing. In some embodiments, the flexible tubing comprises silicon, PVC, PC, or another suitable material.

With reference, to FIG. 26D, a sample collection apparatus is depicted comprising a mixer fluid compartment 2630 having a mixer fluid plunger 2634 disposed within. In some embodiments, the apparatus comprises a physical barrier 2636 provided to stop movement of the mixer fluid compartment 2630 within the housing. In some embodiments, barrier 2636 is formed from protrusions provided in the housing of the apparatus. In some embodiments, the barrier provides a physical stop for the mixer fluid compartment, wherein motion of the mixer fluid compartment is stopped when the cap 2610 of the mixer fluid compartment abuts the barrier 2636. In some embodiments, the barrier 2636 is formed by protrusions extending from the lower and/or upper distal housing portions (e.g. 605 and 611 as depicted in FIG. 6A).

In some embodiments, the arrangement is provided such that when transitioning from a first configuration to a second configuration will first move the mixer fluid compartment 2630 toward the barrier 2636 as it is forced by a protrusion 2613 which moves with a proximal portion of the housing. In some embodiments, the mixer fluid compartment 2630 approaches the barrier 2636, an end of the mixer fluid conduit 2640 pierces a breakable seal provided on the cap 2610 of the mixer fluid compartment 2630. In some embodiments, once the cap 2610 abuts the barrier 2636, motion of the compartment 2630 ceases and the force provided by protrusion 2613 moves plunger 2634 through the compartment to dispense a mixer fluid into and through the mixer fluid conduit 2640.

The barrier and arrangement above may be utilized in any embodiments of the apparatus, as disclosed herein. In some embodiments, the length of the plunger 2634 is about 6 mm. In some embodiments, as the plunger 2634 abuts a tapered end of the mixer fluid compartment 2630, a volume of mixer fluid will be left in the compartment. In some embodiments, about 100 microliters of a mixer fluid will remain within the compartment and or mixer fluid conduit after actuation of the apparatus. In some embodiments, the remaining fluid will have to be accounted for when selecting a volume of mixer fluid to be dispensed. For example, in order to dispense 500 microliters of a mixer fluid, the mixer fluid compartment will be loaded with 600 microliters of a mixer fluid to account for a volume which will remain within the compartment and/or mixer fluid conduit.

### II. Rotational Activation of a Sample Collection Apparatus

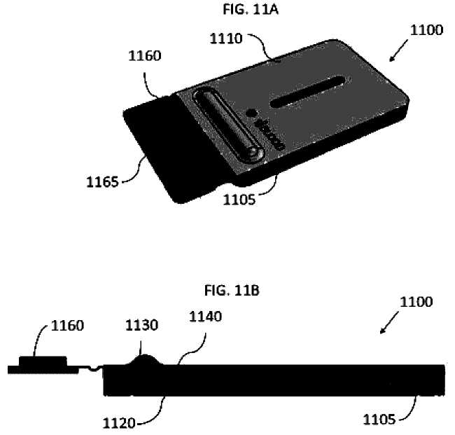

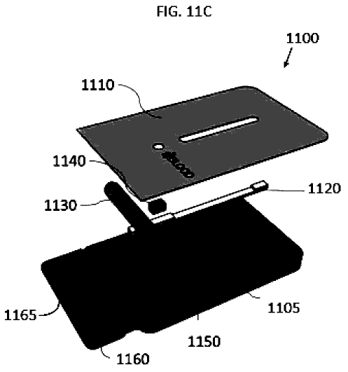

With reference to FIGS. 11A-11C, a sample collection apparatus 1100 is depicted comprising a cover 1110, a housing comprising a main housing 1105 and a moveable portion 1160. In some embodiments, the apparatus further comprises a sample interface 1140, a mixer fluid chamber 1130, a membrane 1120, and a membrane holder 1150 disposed on the main housing 1105. In some embodiments, the membrane holder 1150 is formed by a recess in the housing 1105. In some embodiments, when assembled (as depicted in FIGS. 11A and 11B) the cover 1110 comprises an aperture to provide a sample to a sample interface 1140 and a window to view a membrane 1120 within the housing 1105.

In some embodiments, the apparatus further comprises a moveable portion 1160 after a sample is disposed into the sample interface, a moveable portion 1160 of the housing is configured to be rotated about an axis and be displaced over a portion of the cover 1110, over the mixer fluid compartment 1130 such that the mixer fluid compartment is crushed to release one or more mixer fluids stored within the mixer fluid chamber on to the membrane 1120. In some embodiments, at least a portion of the cover 1110 over the sample chamber 1130 is flexible to facilitate crushing of the chamber 1130. In some embodiments, the moveable portion 1160 comprises a protrusion 1165 to facilitate crushing of the chamber 1130.

In some embodiments, the main housing 1105 and the moveable portion 1160 are connected by a flexible interface or molded hinge. In some embodiments, a material thickness is reduced at the connection between the main housing 1105 and the moveable portion to be rotated 1160 to allow for a bending motion. In some embodiments, the membrane is as described herein, configured for collecting and in some cases, testing a sample (for example, in some cases, the membrane comprises a lateral flow strip assay).

With reference to FIGS. 13A-13C, a sampling apparatus 1301 is depicted comprising a cover 1310 and a main housing 1300. In some embodiments, the apparatus further comprises a sample interface 1360, a mixer fluid compartment 1330, a membrane 1320, and a membrane holder 1350. In some embodiments, the membrane holder 1350 is formed by a recess in the housing 1300. In some embodiments, when assembled (as depicted in FIGS. 13A and 13B) the cover 1310 comprises an aperture to provide a sample to a sample interface 1360 and a window 1390 to view a membrane 1320 within the housing 1300.

In some embodiments, the apparatus further comprises a moveable portion 1340. In some embodiments, after a sample is disposed into the sample interface 1360, the moveable portion 1340 of the housing is rotated about an axis and displaced over the mixer fluid compartment 1330 such that the mixer fluid compartment is crushed to release one or more mixer fluids on to the membrane 1320 via the mixer fluid conduit 1380. In some embodiments, the mixer fluid compartment 1330 is a crushable blister pack. In some embodiments, the moveable portion 1340 comprises a protrusion 1345 to facilitate crushing of the mixer fluid compartment 1330. In some embodiments, the membrane is as described herein, configured for collecting and in some cases, testing a sample (for example, in some cases, the membrane comprises a lateral flow strip assay).

In some embodiments, the cover 1310 and the moveable portion 1340 are connected by a flexible interface or molded hinge. In some embodiments, a material thickness is reduced at the connection between the cover 1310 and the portion of the housing to be rotated 1340 to allow for a bending motion. In some embodiments, the moveable portion 1340 comprises one or more locking tabs 1344 to lock the moveable portion 1340 onto the housing 1300. In some embodiments, the moveable portion 1340 will block access to the sample interface 1360 after actuation.

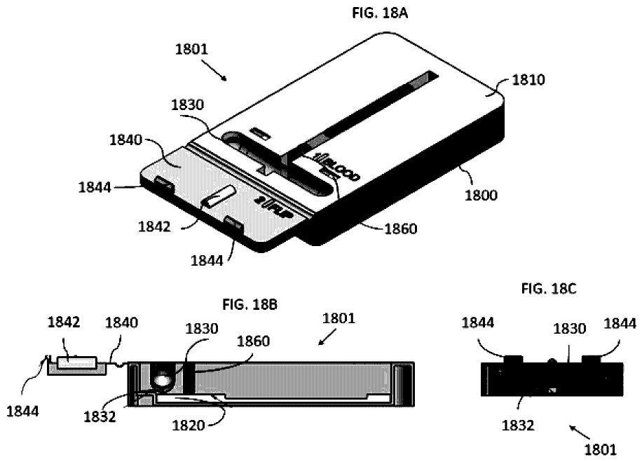

With reference to FIGS. 18A-18C, a sampling apparatus 1801 is depicted comprising a cover 1810 and a housing 1800. In some embodiments, the apparatus further comprises a sample interface 1860, a mixer fluid compartment 1830, and a membrane 1820. In some embodiments, the cover 1810 comprises an aperture to provide a sample to a sample interface 1860. In some embodiments, the housing comprises a window 1890 to view a membrane 1820 within the housing 1800.

In some embodiments, after a sample is disposed into the sample interface 1860, a portion 1840 of the housing is rotated about an axis and displaced over the mixer fluid compartment 1830 such that the mixer fluid compartment is crushed to release one or more mixer fluids on to the membrane 1820. In some embodiments, the mixer fluid compartment 1830 is a glass ampule. In some embodiments, the portion of the housing to be rotated 1840 comprises a protrusion 1842 to facilitate crushing of the mixer fluid compartment 1830. In some embodiments, a space 1832 is provided between the center of the mixer fluid compartment 1830 and the housing 1800 to facilitate breaking of the mixer fluid compartment 1830.

In some embodiments, the housing 1800 and the portion of the housing to be rotated 1840 are connected by a flexible interface or molded hinge. In some embodiments, a material thickness is reduced at the connection between the housing 1800 and the portion of the housing to be rotated 1840 to allow for a bending motion. In some embodiments, the portion to be rotated comprises one or more locking tabs 1844 to be received by one or more recesses 1846 in the housing for locking the portion to be rotated onto into the housing. In some embodiments, the portion to be rotated 1840 will block access to the sample interface 1860 after actuation.

In some embodiments, the membrane is as described herein, configured for collecting and in some cases, testing a sample (for example, in some cases, the membrane comprises a lateral flow strip assay).

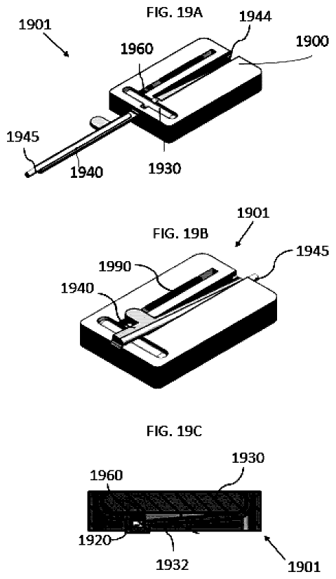

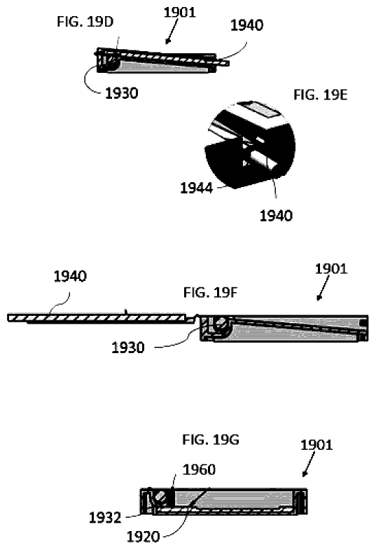

With reference to FIGS. 19A-19G, a sampling apparatus 1901 is depicted, wherein the apparatus 1901 comprises a sample interface 1960, a mixer fluid chamber 1930, a membrane 1920, and a housing 1900. In some embodiments, the apparatus comprises a flexible cover retain the mixer fluid compartment 1930

In some embodiments, after a sample is disposed into the sample interface 1960, a moveable portion 1940 of the housing is rotated about an axis and disposed over the mixer fluid compartment 1930 such that the mixer fluid compartment is crushed to release one or more mixer fluids on to the membrane 1920. In some embodiments, the mixer fluid compartment 1930 is a glass ampule. In some embodiments, the portion of the housing to be rotated 1940 comprises rigid rod 1945 to provide leverage and facilitate crushing of the mixer fluid compartment 1930. In some embodiments, a space 1932 is provided between the center of the mixer fluid compartment 1930 and the housing 1900 to facilitate breaking of the mixer fluid compartment 1930. FIG. 19C provides a front view of the sample apparatus.

In some embodiments, the housing 1900 and the portion of the housing to be rotated 1940 are connected by a flexible interface or molded hinge. In some embodiments, a material thickness is reduced at the connection between the housing 1900 and the portion of the housing to be rotated 1940 to allow for a bending motion. In some embodiments, the housing to be rotated comprises one or more locking tabs 1944 for locking the portion to be rotated onto into the housing. In some embodiments, the portion to be rotated 1940 will block access to the sample interface 1960 after actuation. In some embodiments, the sample conduit 1960 forms a barrier to prevent glass shards from getting onto the membrane.

In some embodiments, the membrane is as described herein, configured for collecting and in some cases, testing a sample (for example, in some cases, the membrane comprises a lateral flow strip assay).

### III. Additional Embodiments of a Sample Collection Apparatus

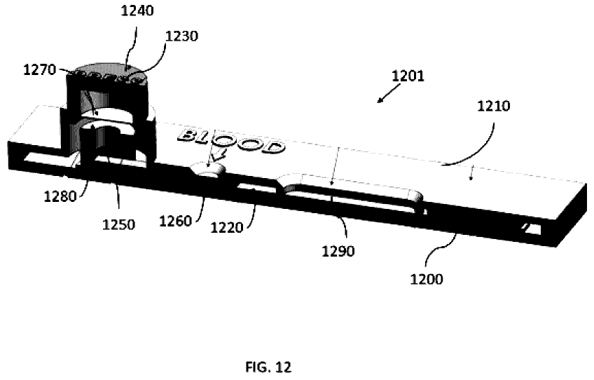

With reference to FIG. 12, a sample collection apparatus 1201 is depicted, according to some embodiments. According to some embodiments, the apparatus 1201 comprises a cover 1210, a housing 1200, a sample interface 1260, and a membrane 1220. In some embodiments, the sample collection apparatus 1201 comprises a depressible button 1240 for breaking a seal 1270 of a mixer fluid chamber 1230. In some embodiments, the apparatus comprises a protrusion 1280 for rupturing the seal 1270. In some embodiments, the seal comprises foil. In some embodiments, the apparatus comprises a mixer fluid conduit 1250 for directing one or mixer fluids from the mixer fluid compartment to the membrane 1220. In some embodiments, the one or more mixer fluids is configured to force the sample to flow across the membrane onto a collection region and/or a testing region, as described herein. In some embodiments, the membrane is as described herein, configured for collecting and in some cases, testing a sample (for example, in some cases, the membrane comprises a lateral flow strip assay).

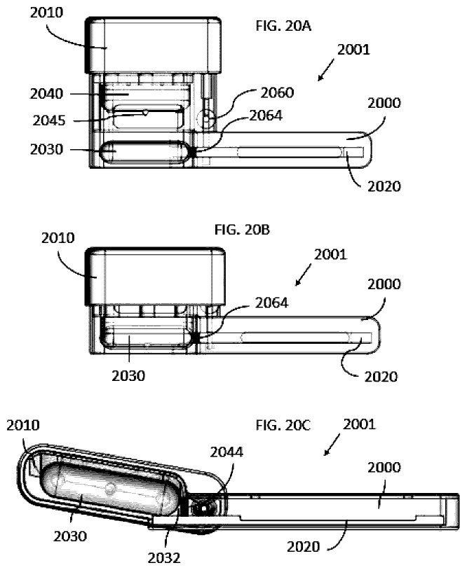

With reference to FIGS. 20A-20C, a sample collection apparatus 2001 is depicted, according to some embodiments. In some embodiments, the collection apparatus comprises a housing 2000, a cover 2010, a sample interface 2060, a mixer fluid chamber 2030, and a membrane 2220. In some embodiments, the sample collection apparatus comprises an actuator 2040 which is engaged by depressing the cover 2010 toward the housing 2000. In some embodiments, depression of the cover 2010 engages and translates a sample plunger within the housing to dispense the sample out of the sample conduit 2064 and onto the membrane 2020. In some embodiments, depression of the cover 2010 pushes a protrusion 2045 of the actuator 2040 into the mixer fluid compartment 2030, thereby causing the mixer fluid compartment to rupture or break. In some embodiments, sample conduit 2064 also serves as a barrier to prevent glass shards of a mixer fluid compartment formed from a glass ampule from getting onto a portion of the membrane.

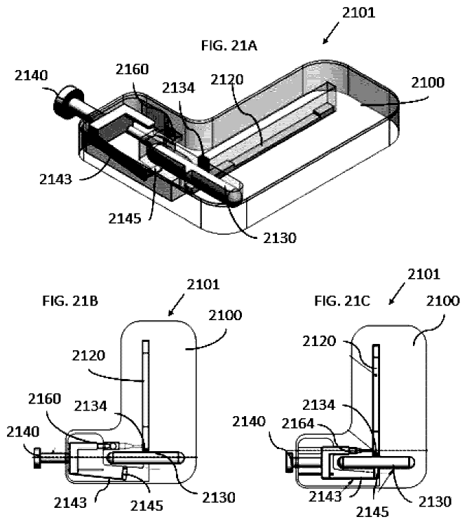

With reference to FIGS. 21A-21C, a sample collection apparatus 2101 is depicted, according to some embodiments. In some embodiments, the collection apparatus comprises a housing 2100, a sample interface 2160, a mixer fluid compartment 2130, and a membrane 2120. In some embodiments, the sample collection apparatus comprises an actuator 2140. In some embodiments, the actuator is configured to be engaged, such that a sample plunger dispenses the sample out of the sample conduit 2164 and onto the membrane 2120.

In some embodiments, the sample collection apparatus comprises a hammer 2145 at the end of a lever 2143. In some embodiments, upon engaging the actuator 2440, the hammer 2145 is forced into the sample chamber 2130 to rupture the mixer fluid chamber. In some embodiments, the sample chamber is a glass ampule. In some embodiments, barrier 2134 prevents glass from getting onto the membrane 2120. In some embodiments, a spring providing a force to the hammer comprises a spring constant of about 5 to 7 pounds per inch. In some embodiments, the weight of the hammer is about 5 to 10 grams.

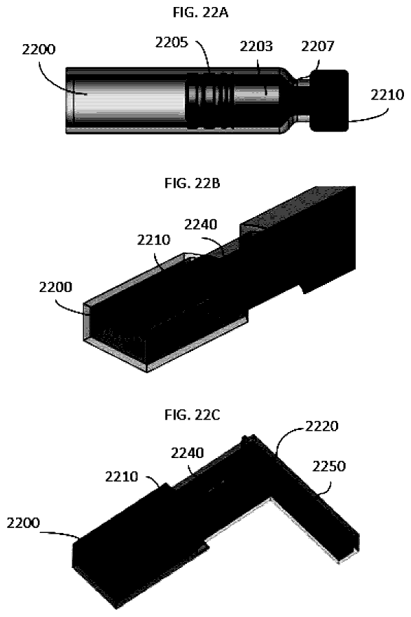

With reference to FIGS. 22A-22C, a mixer fluid compartment 2220 for a sample collection apparatus described herein is depicted. In some embodiments, the mixer fluid compartment comprises a plunger 2205 and a cap 2210. In some embodiments, the mixer fluid compartment comprises a first volume 2203 and a second volume 2207. In some embodiments, the first volume comprises one or more mixer fluids and is dispensed first. In some embodiments, the second volume comprises a filler fluid or low cost liquid for pushing all the mixer fluids out of the mixer fluid compartment. In some embodiments, the second volume of fluid is approximately equal to the volume of the portion of the cap unreachable by the plunger and the volume of the mixer fluid conduit. In some embodiments, this configuration allows all the mixer fluid to dispensed onto the membrane, but the additional fluid remains within the mixer fluid compartment and/or the mixer fluid conduit.

In some embodiments, as depicted by FIG. 22B, a non-coring needle 2240 is used to pierce membrane surface of a cap 2210 of a mixer fluid compartment. In some embodiments, as depicted by FIG. 22C, a fluid channel 2250 is provided perpendicular to a longitudinal axis of a proximal portion 2200 of the apparatus housing. In some embodiments, the fluid channel 2250 is provided perpendicular to a longitudinal axis of a distal portion 2220 of the housing. In some embodiments, the fluid chamber 2250 stores the mixer fluids and sample. In some embodiments, the fluid chamber 2250 retains a membrane for storing a sample or a test to detect or quantify one or more target analytes.

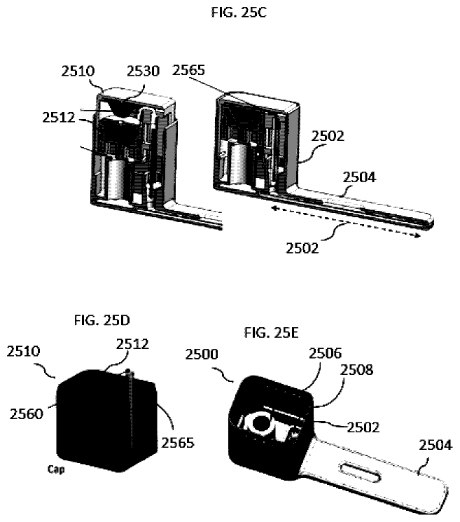

With reference to FIGS. 25A-25E, a sample collection apparatus 2501 is depicted, according to some embodiments. In some embodiments, the sample collection apparatus 2501 comprises a cap for collecting a biological sample 2510 and a base 2500. For consistency, the cap 2510 may refer to the proximal portion or proximal housing, and the base 2500 may refer to the distal portion or distal housing (as used herein). In some embodiments, the cap 2510 and the base 2500 are configured to be detachably coupled to each other (see for FIG. 25B for example). In an embodiment, the cap 2510 comprises a sample interface 2560 formed at one end of a capillary tube 2565. In some embodiments, any other type of conduit may be used instead of or in addition to a capillary tube 2565. In some embodiments, the sample is configured to be received by the capillary tube 2565 at the sample interface 2560 in the cap 2510. In some embodiments, the end of the capillary tube opposite of the sample interface is sealed. In some embodiments, the end of the capillary tube opposite of the sample interface is not sealed when the cap is inserted within the base (as described herein). In some embodiments, the cap 2510 further comprises a mixer fluid chamber 2530 holding one or more mixer fluids therein. In some embodiments, the mixer fluid chamber comprises a breakable seal that rupturable to release the mixer fluids therefrom. In some embodiments, the mixer fluid compartment 2530 is a blister pack holding the one or more mixer fluids. In some embodiments, the sample is received by the cap 2510 (as described herein), wherein the cap 2510 is separated from the base 2500. In some embodiments, after a sample has been received by the cap 2510 (example, received within the capillary tube 2565), the cap 2510 is received by the base 2500. For example, in some embodiments, the base 2500 comprises a receiver 2502 and a membrane housing 2504. In some embodiments, the cap 2510 is configured to be at least partially inserted within the receiver 2502. In some embodiments, the cap comprises a plunger 2512. In some embodiments, the base 2500 comprises a protrusion 2506 disposed within the receiver 2502 of the base 2500. FIG. 25C provides an exemplary depiction of the cap 2510 being inserted within the receiver 2502 of the base 2500. In some embodiments, the apparatus 2501 comprises a first configuration when the cap 2510 is not inserted within the base 2500, and a second configuration when the cap 2510 is inserted within the base 2500. In some embodiments the membrane housing 2504 comprises a membrane (e.g., see 2520) as described herein, for collection and/or testing of a biological sample. For example, in some embodiments, the membrane 2520 comprises a lateral flow assay strip as described herein. In some embodiments, the membrane (e.g., sample pad) extends into the receiver 2502 to receive the biological sample (and in some cases, the mixer fluid, as described herein). In some embodiments, the receiver comprises a receiving port 2508 that provides fluid communication between the capillary tube 2565 and the membrane when the cap 2510 is inserted within the receiver 2502.

In some embodiments, insertion of the cap 2510 within the receiver 2502 permits the biological sample to be displaced through the capillary tube 2565 and onto the membrane. In some embodiments, insertion of the cap within the receiver, as shown in the right image of FIG. 25C, pushes a volume of air that pushes the biological sample through the capillary tube. In some embodiments, insertion of the cap 2510 within the receiver of the base permits the protrusion 2506 to contact the plunger 2512. In some embodiments, the plunger the plunger is moveable within the cap 2510, such that interaction with the protrusion 2506 as the cap 2510 is inserted within the receiver 2502 permits axial movement of the plunger 2506 towards the mixer fluid chamber 2530. In some embodiments, insertions of the cap 2510 within the receiver 2502 permits the plunger 2512 to rupture the breakable seal of the mixer fluid chamber 2530, such that the one or more mixer fluids in the mixer fluid chamber are dispensed onto the membrane 2520. In some embodiments, insertion of the cap 2510 within the receiver 2502 of the base 2500 permits the mixer fluid chamber 2530 to be in fluid communication with the capillary tube 2565, thereby enabling the one or more mixer fluids flow through the capillary tube 2565 to arrive at the membrane 2520. In some embodiments, the base comprises a window 2590 to view results of a test region on the membrane 2520 within the housing. In some embodiments, the one or more mixer fluids is configured to force the sample to flow across the membrane onto a collection region and/or a testing region, as described herein. In some embodiments, the membrane is as described herein, configured for collecting and in some cases, testing a sample (for example, in some cases, the membrane comprises a lateral flow strip assay). In some embodiments, the base 2504 comprises a longitudinal axis 2509. In some embodiments, the cap 2510 is configured to be inserted within the receiver 2502 along a lateral axis that is perpendicular or substantially perpendicular to the longitudinal axis 2509. In some embodiments, cap 2510 is threaded onto the receiver 2502. In some embodiments, the threads are one-way threads or ratcheting threads to prevent removal of the cap.

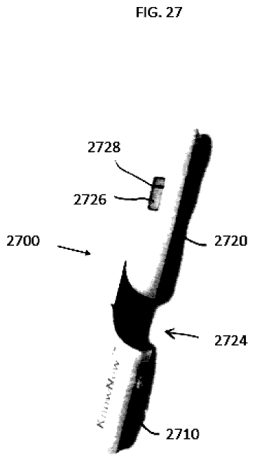

FIGS. 27-31B depict exemplary sample collection apparatuses, as discussed herein, to provide for collection and manipulation of biological samples. In general, the apparatuses, as described herein, may be used to collect a sample, such as a blood sample, and transfer it to a lateral flow strip or a collection membrane. In some embodiments, the apparatuses depicted in FIGS. 27-31B comprise two component parts, a collection body piece, and a cap. The collection piece includes a sample conduit (e.g., a capillary tube) with an exposed end. In some embodiments, the collection piece is held by the patient (or a caregiver) in the hand and the exposed end of the capillary tube is used to collect a biological sample (e.g., blood), such as from a fingertip. The cap, containing a reagent, a diluent, a buffer solution, or some other stored fluid, is then placed over the capillary end of the collection piece. Placing the cap over the end of the collection piece displaces the fluid stored in the cap, forcing the stored fluid into the capillary tube and as a result pushing both of the biological sample and the stored fluid out of the other end of the capillary tube onto the lateral flow strip or membrane.

FIG. 27 depicts a sample collection apparatus 2700 comprising a cap 2710 sized to fit over an end 2724 of a collection body piece 2720, according to some embodiments. In some embodiments, the collection piece 2720 includes a window 2726 that enables viewing a collected sample and/or test result 2728.

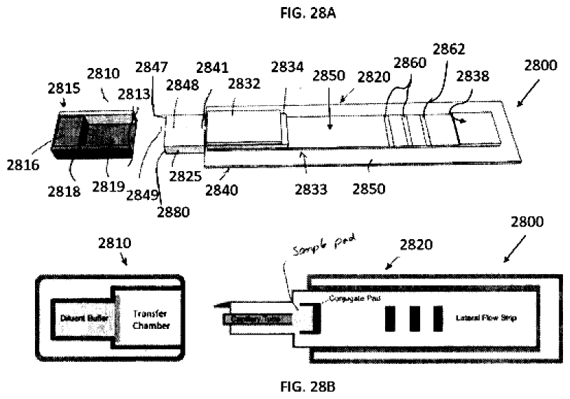

FIGS. 28A and 28B depict a sample collection apparatus 2800 comprising a cap 2810 sized to fit over an end 2825 of a collection body piece 2820, according to some embodiments. In some embodiments, the collection piece 2820 includes a window 2826 that enables viewing a collected sample and/or test result 2828.

In some embodiments, the cap 2810 includes a section 2815 that holds a stored fluid 2816 such as a diluent, a buffer, or some other agent.

In some embodiments, a pierceable membrane 2818 seals and separates the stored fluid 2816 from a transfer chamber area 2819.

In some embodiments, the collection piece 2820 includes one or more media disposed on a substrate 2840 and a support section 2845 that provides support for a capillary tube 2848. In some embodiments, the media 2840 is a lateral flow immunoassay strip 2830. However, as will be discussed below in more detail, other types of media may be used.

In some embodiments, the lateral flow strip 2830 processes captured blood molecules and exposes them to a surface that binds to analytes. In some embodiments, the analytes can then be bound by a conjugate to make them detectable. The bound analytes may also modify the optical or electrical properties of the surface they are bound to, making them directly detectable via visual inspection, or electronic circuits. A window in the housing can provide easy access to review test and optional control lines.

In some embodiments, the lateral flow immunoassay strip 2830 comprises one or more colored nanoparticles (or labels) and antibodies. Example assays include diagnostic assays and chemical detection assays. Diagnostic assays may include, without limitation, lateral flow assays, and may include one or more serological assays. In some embodiments to perform the assay(s), a collected sample flows from the capillary 2848 onto a sample pad 2832, through a conjugate pad 2834, to a membrane 2836 and from there onto an absorbent pad 2838.

In some embodiments, the sample pad 2832 acts as a first stage. In some embodiments, the sample pad 32 contains a filter. In some embodiments, the filter facilitates accurate and controlled flow of the sample through the rest of the apparatus. In some embodiments, the conjugate pad 2834 stores the conjugated labels and antibodies. In some embodiments, the sample exits from the sample pad 2832 and onto the conjugate pad 2834. In some embodiments, if a target analyte is present in the sample, the immobilized conjugated antibodies and labels bind to the target and continue to flow along a membrane 2836. In some embodiments, the membrane is a cellulose substrate. In some embodiments, binding reagents situated on the membrane bind to the target at one or more test lines or test regions 2860. In some embodiment, a colored line will form at the test regions upon detection of the target analyte. In some embodiments, the density or intensity of the test line will vary depending on the quantity of the target present. In some embodiments, any remaining sample passes on to the absorbent pad to collect any excess. In some embodiments, a collection apparatus further comprises a control line 2862 to confirm the apparatus is operating properly.

In some embodiments, the sample is first introduced into the apparatus via an exposed “entrance” end 2849 of the capillary 2848. In some embodiments, by directing the exposed end 2849 of capillary tube 2848 to collect blood droplets from a fingertip. In some embodiments, the capillary 2848 then extract(s) a metered amount of blood. In some embodiments, the apparatus comprises more than one capillary or other type of microfluidic channel(s). In some embodiments, a single sample interface

In some embodiments, the cap 2810 is then placed over the collection piece 2820, such that the capillary tube 2848 and support 2845 fit into the transfer chamber 2819. In some embodiments, a protrusion 2847 on the end of the collection piece is arranged to rupture the membrane 2818, releasing and/or exposing the stored fluid 2816 (which has now been released into the transfer chamber 2819) to also be collected by the capillary 2848. In some embodiments, the action of connecting the cap 2810 to the collection piece 2820 further causes displacement of the blood sample and the stored fluid 2816 from the inboard “exit” end 2841 of capillary 2848. In some embodiments, a seal 2813 may be disposed on the periphery of the cap 2810 and/or collection piece 2820 to ensure that any released fluid does not leak out of the apparatus.

In some embodiments, the blood sample and mixed fluid 2816 then flow onto the sample pad 2832 of the lateral flow strip and then are further pushed into the assay region. This may then enable the lateral flow strip to run one or more tests on the sample as designed and provide results either visually via the test lines 2860 and control lines 2862, or through another detection system such as a reader.

In some embodiments, the fluid 2816 stored in the cap 2810 is a gas such as air, which may or may not be pressurized. In some embodiments, the stored gas may assist with forcing the collected blood sample out of the exit end 2841 of the capillary tube 2848.

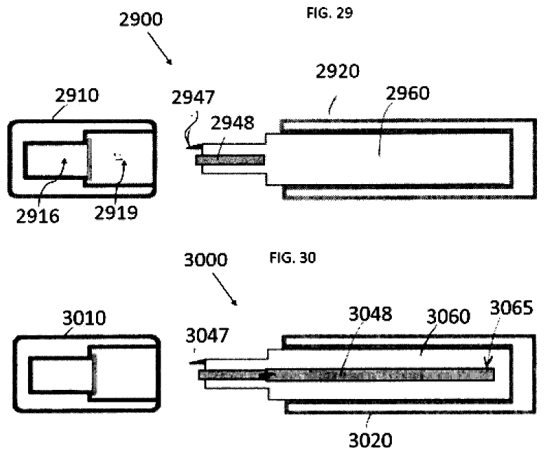

FIG. 29 depicts a sample collections apparatus 2900 which may be considered a variation of the sample collection apparatus 2800 from FIG. 28. In some embodiments, the sample collection apparatus 2900 comprises a sample storage membrane 2960 instead of a lateral flow strip. In some embodiments, the action of connecting the cap 2910 to the collection piece 2920 displaces the blood sample from the capillary 2948 onto the membrane 2960. A membrane 2960, or other sample storage/transport matrix, may be designed to dry out the sample for stable transport at a wide range of temperatures. Various types of membranes such as those based on plastic or glass, or microfluidic detectors may be used.

In some embodiments, the media includes one or more membranes, lateral flow immunoassay strips, or other substrates. For example, the media may include both a collection membrane and an immunoassay lateral flow strip. The collection membrane(s) and immunoassay strip(s) may be disposed parallel to one another, adjacent an outlet of the capillaries. Thus, while not shown in the drawings, in some embodiments, one or more collection membrane(s) may receive and store a blood sample from one or more capillaries, and an immunoassay (or other test) strip(s) may receive and process a blood sample provided from the same of other capillaries.

FIG. 30 depict a sample collection apparatus 300 where the capillary 3048 is elongated and extends to a location closer an opposite end 3065 of the collection piece 3020, according to some embodiments. This arrangement may permit collection of a larger amount of the blood sample.

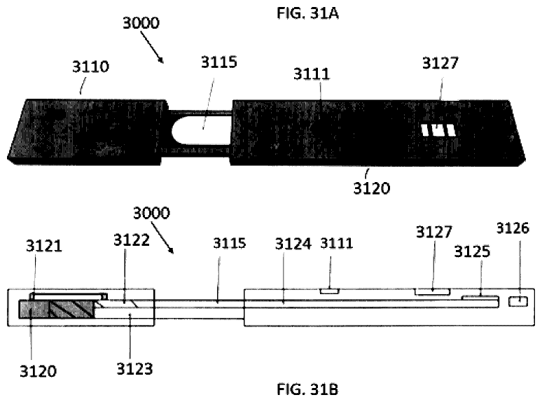

FIGS. 31A and 31B depict a collection apparatus 3100, comprising a lateral flow strip. In some embodiments, a sample collection port 3115 provides a location for collecting a blood sample and a fill window 3111 provides visual feedback as to whether a sufficient amount of sample has been introduced into the apparatus. In some embodiments, the apparatus comprises fluid channel 3121 that connects the liquid mixer fluid reservoir 3120 with the sample collection port once the cap is placed on the apparatus. In some embodiments, the apparatus comprises an empty region 3122 the sample collection port moves into when the apparatus is closed. In some embodiments, the apparatus comprises a rigid support 3123 underneath the lateral flow strip that extends into the liquid mixer fluid portion of the housing. In some embodiments, the apparatus comprises a lateral flow strip 3124. In some embodiments, the apparatus comprises a sample absorbent pad 3125 at the end of the lateral flow strip. In some embodiments, the apparatus comprises a desiccant tab 3126. In some embodiments, the apparatus comprises a window 3127 to view a test region of the lateral flow strip 3124.

In some embodiments, to complete a self-administered collection of a blood sample using the collection apparatus, a subject submitting the sample has pricked a fingertip on the on a hand with a lancet. In some embodiments, the subject grasps the collection apparatus collection apparatus in the other had (not pricked), and with the exposed end of the capillary tube pointing downwards towards the tip of a finger on the pricked hand, blood is drawn into the capillary tube. Once the blood sample is collected, the patient/subject then places the cap over the collection apparatus, as previously described.

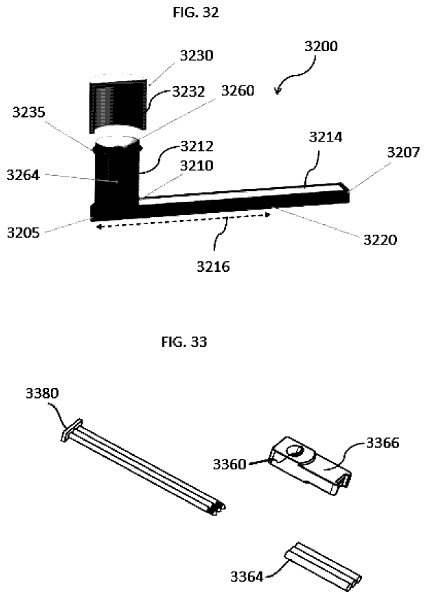

With reference to FIG. 32, an apparatus 3200 for collection of a blood sample is depicted according to some embodiments. In some embodiments, the apparatus comprises a base 3205 mates with housing 3210 to provide a friction or interference fit, such that an outer surface of the sidewalls of the housing 3210 fit snugly against an inner surface of the sidewalls 3207 of the base 3205. In some embodiments, the friction fit retains the housing 3210 onto the base 3205. In some embodiments, a molded hinge at one end connects the base 3205 to the housing 3210.

In some embodiments, a sample conduit 3264 is provided with a sample interface 3260 (i.e. opening or aperture to receive a sample) at a proximal end of the tube. In some embodiments, the sample conduit 3264 is a capillary tube. In some embodiments, the sample conduit 3264 is in fluid communication with the sample interface 3260. In some embodiments, the sample conduit 3264 provides a fluid path for a sample into a storage chamber 3220. In some embodiments, a membrane or lateral flow assay is provided in the storage chamber 3220. In some embodiments, the housing 3210 comprises a lateral portion 3212 and a longitudinal portion 3214. In some embodiments, the lateral portion 3212 comprises the sample interface 3260 and sample conduit 3264, while the longitudinal portion 3214 comprises the storage chamber 3220.

In some embodiments, the lateral portion 3212 of the housing comprises a circular recess to retain a sealing ring 3235 (i.e. an O-ring) thereon. In some embodiments, the lateral portion 3212 is configured to be at least partially inserted within a cap 3230. In some embodiments, the sealing ring provides an airtight seal between the proximal end of the housing and the cap 3230. Accordingly, in some embodiments, insertion of the lateral portion 3212 within the cap 3230 provides a positive displacement force via air (for example) disposed within a region 3232 of the cap as the lateral portion 3212 is being inserted therein. In some embodiments, the positive displacement force permits a biological sample collected via the sample interface 3260 to be dispensed through the conduit 3264 and into the storage chamber. In some embodiments, the conduit 3264 and/or the storage chamber 3220 comprises one or more reagents configured to interact with the biological sample. In some embodiments, the one or more reagents comprises heparin, and/or etheylenediaminetetraacetic acid (EDTA). In some embodiments, the longitudinal portion 3214 comprises a longitudinal axis 3216. In some embodiments, the lateral portion 3212 is configured to be inserted within the cap 3230 along a lateral axis that is perpendicular or substantially perpendicular to the longitudinal axis 3216.

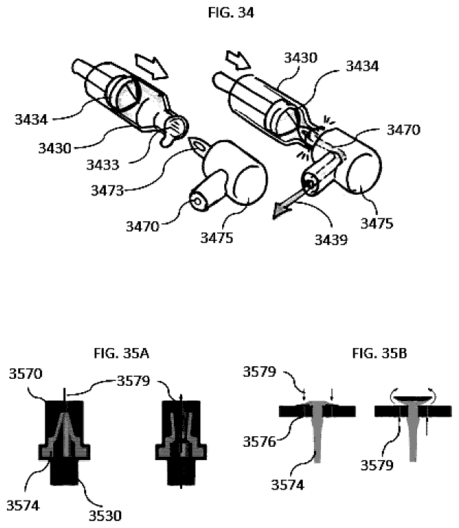

With reference to FIG. 34 depicts a connector 3475 comprising a mixer fluid conduit 3470 configured to interface with a mixer fluid compartment 3430 to transport a mixer fluid, according to some embodiments. In some embodiments, the connector 3475 is configured to engage a mixer fluid compartment retaining a volume of mixer fluid, as disclosed herein. In some embodiments, an end 3473 of the mixer fluid conduit 3470 is tapered to form a piercing element. In some embodiments, the mixer fluid compartment 3430 comprises a breakable seal, as disclosed herein. In some embodiments, the breakable seal 3433 is punctured by the end 3473 of the mixer fluid conduit. The breakable seal 3433 may comprise a foil seal, as depicted.

In some embodiments, as the sample collection apparatus, as described herein, moves from a first to a second configuration, mixer plunger 3434 pushes the mixer fluid compartment toward the connector 3475. In some embodiments, an end 3473 of the mixer fluid conduit 3470 punctures the breakable seal 3433. In some embodiments, mixer fluid within the mixer fluid compartment begins to flow into the mixer fluid conduit 3470 as a pressure is provided by the mixer plunger 3434 to cause a fluid flow 3439 of the mixer fluid through the conduit 3470

In some embodiments, an end of the mixer fluid compartment abuts the connector. In some embodiments, a seal is formed at the mating between an end of the compartment 3430 and the connector 3475. In some embodiments, the connector comprises an inner diameter approximately equal to an outer diameter of the end of the fluid compartment received by the connector to form a seal.

With reference to FIGS. 35A and 35B, exemplary configurations of a one-way valve, which may be utilized in the sample collection apparatus (as disclosed herein) are depicted. In some embodiments, the valves are provided at an interface between a mixer fluid compartment 3530 and a mixer fluid conduit 3570, as disclosed herein. The valves may allow for a needless design, replacing the need for an end of the mixer fluid conduit to be tapered to form a piercing element.

In some embodiments, as depicted in FIG. 35A, in some embodiments, a one-way valve 3574 is provided to limit a fluid flow 3579 to one direction. For example, the valve 3574 may be used to prevent a fluid flow from a mixer fluid conduit 3570 back into the mixer fluid compartment 3530. In some embodiments, the valve 3574 comprises an elastic material which is tapered toward one end. The taper is in the direction of the desired fluid flow as depicted. In some embodiments, the taper is formed by a conical section of the valve. In some embodiments, the taper is formed by two sections which are pressed together by an elastic force (i.e. a reed valve). In some embodiments, when a fluid flow exerts a pressure through the valve, the tapered end expands to allow the mixer fluid to flow through the valve.

FIG. 35B depicts another configuration of a fluid valve, according to some embodiments. In some embodiments, valve 3574 comprises flaps to seal apertures 3576 provided through a surface. In some embodiments, the valve 3574 comprises an elastic material, such that when the flaps of the valve bend away from the apertures when a fluid flow 3579 is provided in a desired direction. The fluid valve depicted by FIG. 35B may be integrated, for example, at an interface between a mixer fluid compartment and a mixer fluid conduit. In some embodiments, the fluid valve is integrated into a cap of a mixer fluid compartment.

### IV. Automated Test Region Analysis

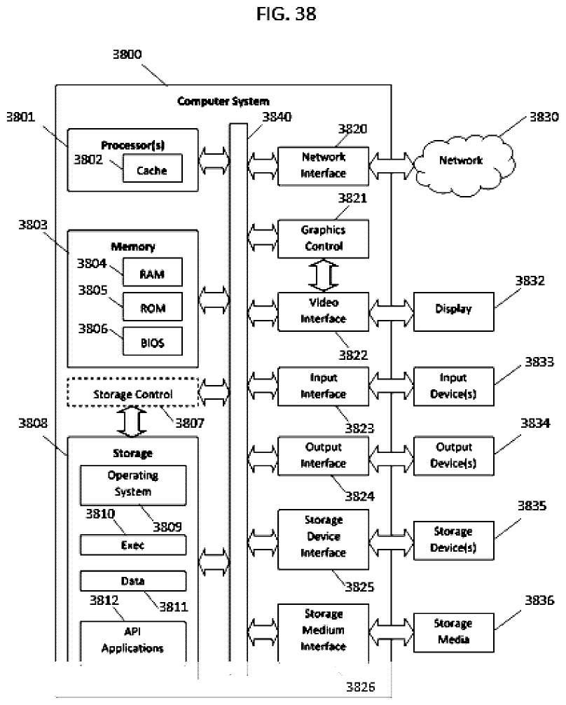

In some embodiments, a computer system herein is utilized to analyze a test region of a lateral flow strip. FIG. 37 depicts an exemplary test region 3720 of a lateral flow assay provided on a membrane of within a sample collection apparatus, as disclosed herein. In some embodiments, test region 3720 comprises a plurality of detection regions (3721, 3722, 3723, 3724, 3725, 3728, 3729) for detecting target analytes. In some embodiments, each detection region corresponds to a different analyte type. In some embodiments, each detection region corresponds to a different variant of a pathogen. In some embodiments, one or more control detection regions 3728, 3729 are provided in the test region to ensure the biological sample has been properly dispersed through the test region. In some embodiments, a first control region 3728 is provided at a first end of the test region and a second control region 3729 is provided at an opposite end of the test region to ensure the biological sample has been properly dispersed through the test region.

In some embodiments, an image of the test region is captured by one or more image sensors. In some embodiments, images of the test region are analyzed by a computing system. In some embodiments, the computing system process the images and outputs a corresponding signal intensity value for each of the detection regions. In some embodiments, a detection threshold is set such an analyte will be considered present in the sample if intensity of the corresponding detection region is above the detection threshold value. In some embodiments, the intensity of the detection regions is corresponded directly to a quantification (e.g. a concentration) of an analyte within the sample.

FIG. 37 depicts a graphical representation 3750 of an intensity analysis performed by a computing system. In some embodiments, an intensity threshold 3755 is set such that any intensity signals above the threshold output a detection of an analyte corresponding of the corresponding detection region. In some embodiments, a quantification of an analyte within a sample corresponds to the area under the intensity curve corresponding to the detection region of said analyte. In some embodiments, a quantification of an analyte within a sample corresponds to the area under the intensity curve, but above the threshold 3755, corresponding to the detection region of said analyte.

### V. Antibody Dashboard

In some embodiments, provided herein are systems and methods for monitoring an antibody level of a subject or group of subjects. In some embodiments, systems and methods include monitoring a group of subjects to determine an overall antibody level of the group. In some embodiments, the group is identified by common attribute.

In some embodiments, antibody levels of a subject are transmitted to one or more databases via a computing apparatus. In some embodiments, a software application is loaded onto the computing apparatus. In some embodiments, the computing apparatus comprises at least one image sensor. In some embodiments, the computing apparatus comprises a camera. In some embodiments, a subject provides a biological sample into a sample collection apparatus (for example, an apparatus described herein), comprising a testing region with a window to view said testing region. For example, the testing region may be configured to detect one or more analytes corresponding to one or more antibodies for a pathogen or virus. As described herein, in some embodiments, the detecting may be visible via the testing region via a color change, fluorescence emitted, or other visual signal. In some embodiments, the results of a sample deposited into a testing region is manually loaded onto the computing apparatus (e.g., if an antibody is detected or not detected). In some embodiments, the results of the sample are deposited into a testing region is captured as one or more images a computing apparatus using an image sensor. In some embodiments, the images of the test results are transmitted to a computing system for analysis. In some embodiments, analysis of the test result images is automated. In some embodiments, analysis includes a determination of the presence of one or more target analytes. In some embodiments, analysis further includes a quantification of a concentration of one or more target analytes present in the biological sample. In some embodiments, the results of the analysis are stored in one or more databases of the computing system.

In some embodiments, a biological sample is provided to a collection apparatus, as disclosed herein, and the collection apparatus is shipped to a laboratory for analysis or submission of test results to a user database.

In some embodiments, a software application loaded on a storage medium of the user computing apparatus facilitates transmission of the captured images of the test results to the computing system. In some embodiments, a web or cloud based software application facilitates, accessible by the user computing apparatus, facilitates transmission of the captured images of the test results to the computing system, analysis of the captured images, transmission of data to a database of the computing system, or a combination thereof. In some embodiments, the software application gather's patient information. In some embodiments, patient/subject information is utilized to group results of the analyte tests. In some embodiments, grouping of subjects is based on age, gender, race/ethnicity, occupation, vaccine type, location, target analyte levels, pre-disposition to diseases/conditions, pre-existing conditions/diseases, a status, an event, or a combination thereof.

In some embodiments, analysis of biological samples collected by the sample collection apparatus herein are utilized to determine antibody levels of a group. In some embodiments, determined antibody levels of a group are utilized to set safety restrictions. For example, if a group of individual is planning to gather at an event, each individual might provide a biological sample to a sample collection apparatus, as disclosed herein, to provide an analysis of SARS COVID-19 antibody levels. The overall levels of the group may be analyzed to determine safety restrictions for the planned event. For example, if a group analysis determines the group has an antibody level of 95% or more, the event may not require masks and social distancing. group analysis determines the group has an antibody level of 94% or less, the event may require masks and social distancing of at least 6 feet. In an example, if a group has an antibody level of 50-75%, an event may have to take place at a limited capacity. In some embodiments, individuals with low or no antibody levels can be restricted from attending a particular event to comply with a required level of antibodies.

In some embodiments, a plurality of analyses performed by submitting results from or samples using the collection apparatus described herein are utilized to track information such as vaccine effectiveness, herd immunity, infectious disease transfer patterns, etc.

Exemplary application of monitoring number of people not having antibody for a pathogen or virus.

For a sporting event at a stadium, local, state, and/or federal regulations may be imposed to limit the spread and infection of a pathogen or virus. For example, the pathogen may be SARS COVID-19. The regulations may require a threshold level (e.g., proportion) of people located within the stadium contain antibodies for the pathogen/virus. For example, the threshold level may be at least 15% to about 95% of the people located within the stadium at any time. In some cases, the regulations may limit the number of people not containing antibodies into the stadium if the threshold level is not met. In some cases, the regulation may impose the requirement for wearing protective apparel, such as masks, based on the threshold level.

In some cases, each person entering may be required to take a serology test to determine if the person contains antibodies for the pathogen. The serology test may be by using any apparatus described herein (for example, see FIGS. 5A-6H). In some cases, using an apparatus described herein enables for a rapid test to be conducted, wherein results may be provided within 30 seconds to 25 minutes. In some cases, the serology test may be performed at any time before entering the stadium, and may be valid for a prescribed amount of time prior to entering the stadium (e.g., the test may be conducted up to 2 weeks, 2 months, 6 months, 1 year, etc) before the event.

In some cases, each person may be provided with an identification tag or marker to indicate the antibody status (i.e. containing the antibody or not). In some cases, a computing device described herein is configured to store and monitor the proportion of people (or number of people) containing antibodies within the stadium. In some cases, a person leaving the stadium is required to scan the identification tag or marker, such that the computing device or other monitoring system can update the proportion of people (or number of people) containing antibodies within the stadium in real time.

### VI. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory mixer fluid such that it is separated from the biological source from which the material is obtained. In vitro assays can encompass cell-based assays in which living or dead cells are employed. In vitro assays can also encompass a cell-free assay in which no intact cells are employed.

The term “mixer fluid” as used herein may refer to a fluid that contacts with a biological sample received by an apparatus described herein. In some embodiments, the mixer fluid refers to a buffer solution. In some embodiments, mixer fluid comprises one or more reagents configured to interact and induce a chemical interaction with the biological sample. In some embodiments, the mixer fluid comprises one or both of the buffer solution and the one or more reagents. In some embodiments, the buffer solution and/or the mixer fluid permits movement of the collected biological sample through, for example a sample conduit (e.g., capillary tube), and/or permits movement of the biological sample across a membrane a described herein (e.g., lateral flow assay strip). In some embodiments, the buffer solution comprises water, a saline solution, PBS, or others buffer solutions.

The term “mixer chamber”, “mixer compartment”, “mixer fluid chamber”, and “mixer fluid compartment” may be used interchangeably herein, and are configured to hold one or more mixer fluids as part of any apparatus embodiment described herein.

The term distal portion and distal housing may be used interchangeably herein.

The term proximal portion and proximal housing may be used interchangeably herein.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### Computing System

Referring to FIG. 38, a block diagram is shown depicting an exemplary machine that includes a computer system 3800 (e.g., a processing or computing system) within which a set of instructions can execute for causing a device to perform or execute any one or more of the aspects and/or methodologies for static code scheduling of the present disclosure. The components in FIG. 38 are examples only and do not limit the scope of use or functionality of any hardware, software, embedded logic component, or a combination of two or more such components implementing particular embodiments.

Computer system 3800 may include one or more processors 3801, a memory 3803, and a storage 3808 that communicate with each other, and with other components, via a bus 3840. The bus 3840 may also link a display 3832, one or more input devices 3833 (which may, for example, include a keypad, a keyboard, a mouse, a stylus, etc.), one or more output devices 3834, one or more storage devices 3835, and various tangible storage media 3836. All of these elements may interface directly or via one or more interfaces or adaptors to the bus 3840. For instance, the various tangible storage media 3836 can interface with the bus 3840 via storage medium interface 3826. Computer system 3800 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 3800 includes one or more processor(s) 3801 (e.g., central processing units (CPUs), general purpose graphics processing units (GPGPUs), or quantum processing units (QPUs)) that carry out functions. Processor(s) 3801 optionally contains a cache memory unit 3802 for temporary local storage of instructions, data, or computer addresses. Processor(s) 3801 are configured to assist in execution of computer readable instructions. Computer system 3800 may provide functionality for the components depicted in FIG. 38 as a result of the processor(s) 3801 executing non-transitory, processor-executable instructions embodied in one or more tangible computer-readable storage media, such as memory 3803, storage 3808, storage devices 3835, and/or storage medium 3836. The computer-readable media may store software that implements particular embodiments, and processor(s) 3801 may execute the software. Memory 3803 may read the software from one or more other computer-readable media (such as mass storage device(s) 3835, 3836) or from one or more other sources through a suitable interface, such as network interface 3820. The software may cause processor(s) 3801 to carry out one or more processes or one or more steps of one or more processes described or illustrated herein. Carrying out such processes or steps may include defining data structures stored in memory 3803 and modifying the data structures as directed by the software.

The memory 3803 may include various components (e.g., machine readable media) including, but not limited to, a random access memory component (e.g., RAM 3804) (e.g., static RAM (SRAM), dynamic RAM (DRAM), ferroelectric random access memory (FRAM), phase-change random access memory (PRAM), etc.), a read-only memory component (e.g., ROM 3805), and any combinations thereof. ROM 3805 may act to communicate data and instructions unidirectionally to processor(s) 3801, and RAM 3804 may act to communicate data and instructions bidirectionally with processor(s) 3801. ROM 3805 and RAM 3804 may include any suitable tangible computer-readable media described below. In one example, a basic input/output system 3806 (BIOS), including basic routines that help to transfer information between elements within computer system 3800, such as during start-up, may be stored in the memory 3803.

Fixed storage 3808 is connected bidirectionally to processor(s) 3801, optionally through storage control unit 3807. Fixed storage 3808 provides additional data storage capacity and may also include any suitable tangible computer-readable media described herein. Storage 3808 may be used to store operating system 3809, executable(s) 3810, data 3811, applications 3812 (application programs), and the like. Storage 3808 can also include an optical disk drive, a solid-state memory device (e.g., flash-based systems), or a combination of any of the above. Information in storage 3808 may, in appropriate cases, be incorporated as virtual memory in memory 3803.

In one example, storage device(s) 3835 may be removably interfaced with computer system 3800 (e.g., via an external port connector (not shown)) via a storage device interface 3825. Particularly, storage device(s) 3835 and an associated machine-readable medium may provide non-volatile and/or volatile storage of machine-readable instructions, data structures, program modules, and/or other data for the computer system 3800. In one example, software may reside, completely or partially, within a machine-readable medium on storage device(s) 3835. In another example, software may reside, completely or partially, within processor(s) 3801.

Bus 3840 connects a wide variety of subsystems. Herein, reference to a bus may encompass one or more digital signal lines serving a common function, where appropriate. Bus 3840 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 3800 may also include an input device 3833. In one example, a user of computer system 3800 may enter commands and/or other information into computer system 3800 via input device(s) 3833. Examples of an input device(s) 3833 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 3833 may be interfaced to bus 3840 via any of a variety of input interfaces 3823 (e.g., input interface 3823) including, but not limited to, serial, parallel, game port, USB, FIREWIRE, THUNDERBOLT, or any combination of the above.

In particular embodiments, when computer system 3800 is connected to network 3830, computer system 3800 may communicate with other devices, specifically mobile devices and enterprise systems, distributed computing systems, cloud storage systems, cloud computing systems, and the like, connected to network 3830. Communications to and from computer system 3800 may be sent through network interface 3820. For example, network interface 3820 may receive incoming communications (such as requests or responses from other devices) in the form of one or more packets (such as Internet Protocol (IP) packets) from network 3830, and computer system 3800 may store the incoming communications in memory 3803 for processing. Computer system 3800 may similarly store outgoing communications (such as requests or responses to other devices) in the form of one or more packets in memory 3803 and communicated to network 3830 from network interface 3820. Processor(s) 3801 may access these communication packets stored in memory 3803 for processing.

Examples of the network interface 3820 include, but are not limited to, a network interface card, a modem, and any combination thereof. Examples of a network 3830 or network segment 3830 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 3830, may employ a wired and/or a wireless mode of communication. In general, any network topology may be used.

Information and data can be displayed through a display 3832. Examples of a display 3832 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 3832 can interface to the processor(s) 3801, memory 3803, and fixed storage 3808, as well as other devices, such as input device(s) 3833, via the bus 3840. The display 3832 is linked to the bus 3840 via a video interface 3822, and transport of data between the display 3832 and the bus 3840 can be controlled via the graphics control 3821. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 3832, computer system 3800 may include one or more other peripheral output devices 3834 including, but not limited to, an audio speaker, a printer, a storage device, and any combinations thereof. Such peripheral output devices may be connected to the bus 3840 via an output interface 3824. Examples of an output interface 3824 include, but are not limited to, a serial port, a parallel connection, a USB port, a FIREWIRE port, a THUNDERBOLT port, and any combinations thereof.

In addition or as an alternative, computer system 3800 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of information. In various embodiments, suitable databases include, by way of non-limiting examples, relational databases, non-relational databases, object oriented databases, object databases, entity-relationship model databases, associative databases, XML databases, and document oriented databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

While embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An apparatus for detecting an analyte molecule in a biological sample, the apparatus comprising:
a housing having a proximal portion and a distal portion, wherein one or both of the proximal portion and distal portion are moveable so as to transition the housing from a first configuration to a second configuration;
a sample interface disposed within the housing, wherein the sample interface is configured to receive the biological sample when the housing is in the first configuration;
a first conduit configured to receive the biological sample via the sample interface;
a mixer fluid compartment disposed within the housing, wherein the mixer fluid compartment is configured to retain a mixer fluid, wherein the mixer fluid compartment comprises a breakable seal, and wherein the breakable seal is configured to break when the housing is transitioned from the first configuration to the second configuration;
a second conduit disposed within the housing, wherein the second conduit is configured to be in fluid communication with the mixer fluid compartment when the housing is in the second configuration; and
a membrane disposed within the housing, wherein the membrane is in fluid communication with the first conduit and the second conduit, so as to receive the biological sample and the mixer fluid when the housing is in the second configuration, wherein the membrane comprises a testing region configured to permit detection of the analyte molecule.

2. The apparatus of claim 1, further comprising a testing window, wherein the testing window allows visual inspection of the testing region.

3. The apparatus of claim 1, wherein the testing region comprises a first detector molecule configured to emit a first signal via the presence of the analyte molecule.

4. The apparatus of claim 3, wherein the testing region comprises a second detector molecule spaced apart from the first detector molecule, wherein the second detector molecule is configured to emit a second signal via the presence of a second analyte molecule.

5. The apparatus of claim 4, wherein the testing region comprises a third detector molecule spaced apart from the first detector molecule and the second detector molecule, wherein the third detector molecule is configured to emit a third signal via the presence of a third analyte molecule.

6. The apparatus of claim 3, wherein the first detector molecule corresponds to an antibody for a pathogen or a virus.

7. The apparatus of claim 6, wherein the pathogen or virus comprises COVID-19 or a variant of COVID-19.

8. The apparatus of claim 1, wherein changing the housing from the first configuration to the second configuration provides positive displacement pressure for the mixer fluid to move within the first tube.

9. The apparatus of claim 1, wherein a sample plunger disposed within the housing is configured to move the biological sample through the first conduit as the housing is transitioned from a first configuration to a second configuration.

10. The apparatus of claim 1, wherein a mixer plunger disposed within the housing is configured to dispense the mixing fluid into the second conduit as the housing is transitioned from the first configuration to the second configuration.

11. The apparatus of claim 1, wherein the first tube comprises a capillary tube.

12. The apparatus of claim 1, further comprising a sample window, wherein the sample window allows visual inspection of the biological sample within the first tube.

13. The apparatus of claim 1, further comprising a locking tab, wherein the locking tab engages a portion of the housing in the second configuration to prevent movement from the second configuration towards the first configuration.

14. The apparatus of claim 1, further comprising a removeable clip for preventing transition of the apparatus to the second configuration, wherein the removable clip comprises a protrusion to correspond to a recess provided in the housing, wherein mating of the protrusion within the recess facilitates retention of the removable clip on the housing.

15. The apparatus of claim 18, wherein the sample plunger and the mixer plunger are offset, such that the biological sample is dispensed onto the membrane prior to the mixing fluid being dispensed onto the membrane.

16. The apparatus of claim 1, wherein the mixer fluid facilitates the biological sample to flow across the membrane to the testing region.

17. The apparatus of claim 1, wherein the mixer fluid compartment comprises a pierceable membrane.

18. The apparatus of claim 1, wherein said breakable seal is punctured as the housing is transitioned from a first configuration to a second configuration, thereby providing fluid communication between the testing region and the mixer fluid compartment.

19. The apparatus of claim 1, the apparatus is configured to dispense the mixing fluid into the second conduit as the housing is transitioned from the first configuration to the second configuration.

20. A method for detecting an analyte molecule in a biological sample, the method comprising:
(a) providing an apparatus comprising:
a housing having a proximal portion and a distal portion, wherein one or both of the proximal portion and distal portion are moveable so as to transition the housing from a first configuration to a second configuration;
a sample interface located between the proximal portion and distal portion, wherein the sample interface is configured to receive the biological sample when the housing is in the first configuration;
a first conduit configured to receive the biological sample via the sample interface;
a testing region disposed within the housing, wherein the testing region is in fluid communication with the first conduit;
a mixer fluid compartment disposed within the housing, wherein the mixer fluid compartment is configured to retain a mixer fluid, wherein the mixer fluid compartment comprises a breakable seal, and wherein the breakable seal is configured to break when the housing is transitioned from the first configuration to the second configuration;
a second conduit disposed within the housing, wherein the second conduit is configured to be in fluid communication with the mixer fluid compartment when the housing is in the second configuration; and
a testing region disposed within the housing, wherein the testing region is in fluid communication with the first conduit and the second conduit,
(b) receiving the biological sample via the sample interface; and
(c) moving the housing from the first configuration to the second configuration, thereby enabling both the mixer fluid and the biological sample to be transferred to the testing region, wherein the testing region is configured to permit detection of the analyte molecule.
